(19) United States
(12) Patent Application Publication
(10) Pub. No.: US 2024/0245074 A1
(43) Pub. Date: Jul. 25, 2024

(54) PURIFIED PROTEIN COMPOSITIONS AND METHODS OF PRODUCTION

(71) Applicant: Clara Foods Co., Daly City, CA (US)

(72) Inventors: Aniket KALE, Pleasanton, CA (US); Ranjan PATNAIK, Daly City, CA (US)

(73) Assignee: Clara Foods Co., Daly City, CA (US)

(21) Appl. No.: 18/423,024

(22) Filed: Jan. 25, 2024

Related U.S. Application Data
(60) Provisional application No. 63/481,527, filed on Jan. 25, 2023.

(51) Int. Cl. A23J 1/18; A23J 3/04; A23J 3/20; A23L 33/195; C12N 1/16; C12N 15/81

(52) U.S. Cl. CPC A23J 1/18; A23J 3/04; A23J 3/20; A23L 33/195; C12N 1/165; C12N 15/81

(57) ABSTRACT

The present disclosure provides methods for producing consumable recombinant proteins that are substantially free from herein-disclosed undesired byproducts.

### CROSS-REFERENCE TO RELATED APPLICATIONS

This application claims the benefit of and priority to U.S. Provisional Application No. 63/481,527, filed Jan. 25, 2023, which is hereby incorporated by reference in its entirety.

### BACKGROUND

Ideally, compositions of consumable recombination proteins are free from undesired manufacturing ingredients, contaminants, and other microbial components and byproducts. In some instances, a recombinant microbial cell synthesizes measurable amounts of undesired byproducts and these must be isolated from the desired consumable recombination proteins when producing a commercial product. There remains an unmet need to produce consumable recombination proteins that are substantially free from such undesired byproducts.

### SUMMARY

The present disclosure provides methods for producing consumable recombinant proteins that are substantially free from herein-disclosed undesired byproducts.

An aspect of the present disclosure is a method for preparing a consumable composition. The method comprising steps of: obtaining a composition comprising a recombinant protein and a plurality of recombinant cell byproducts, wherein the recombinant cell byproducts comprise an exopolysaccharide (EPS) and an off-flavor component; contacting the composition with an anionic resin that reversibly attaches to the recombinant protein and does not substantially attach to the plurality of recombinant cell byproducts; separating the recombinant protein attached to the anionic resin from the plurality of recombinant cell byproducts; collecting the separated recombinant protein, thereby obtaining a protein product having a reduced quantity of the plurality of recombinant cell byproducts; and formulating a consumable composition comprising the protein product.

Another aspect of the present disclosure is a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises resuspending the dry protein composition in an amphipathic solvent, wherein the amphipathic solvent separates the recombinant protein from the recombinant cell byproduct; collecting the separated recombinant protein, thereby obtaining a protein product having a reduced quantity of the plurality of recombinant cell byproducts; and formulating a consumable composition comprising the protein product.

A further aspect of the present disclosure is a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises one or more cation exchange resins that reversibly attach to the recombinant protein and do not substantially attach to the plurality of recombinant cell byproducts; collecting the separated recombinant protein, thereby obtaining a protein product having a reduced quantity of the plurality of recombinant cell byproducts; and formulating a consumable composition comprising the protein product.

A further aspect of the present disclosure is a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises an anionic resin that reversibly attach to the recombinant protein and do not substantially attach to the plurality of recombinant cell byproducts; collecting the separated recombinant protein, thereby obtaining a protein product having a reduced quantity of the plurality of recombinant cell byproducts; and formulating a consumable composition comprising the protein product.

In an aspect, the present disclosure provides a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises resuspending the dry protein composition and a diafiltration treatment, an ultrafiltration step, and/or microfiltration step of the resuspended dry protein composition; collecting the separated recombinant protein, thereby obtaining a protein product having a reduced quantity of the plurality of recombinant cell byproducts; and formulating a consumable composition comprising the protein product.

In an aspect, the present disclosure provides a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises an oxidation step (e.g., addition of hydrogen peroxide); collecting the separated recombinant protein, thereby obtaining a protein product having a reduced quantity of the plurality of recombinant cell byproducts; and formulating a consumable composition comprising the protein product.

In an aspect, the present disclosure provides a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises maintaining the composition under heat and vacuum conditions; collecting the separated recombinant protein, thereby obtaining a protein product having a reduced quantity of the plurality of recombinant cell byproducts; and formulating a consumable composition comprising the protein product.

In another aspect, the present disclosure provides a consumable composition obtained by any herein disclosed method.

In one aspect, disclosed herein are methods for preparing a recombinant protein concentrate for a consumable food product, the protein product having a reduced quantity of recombinant cell byproducts, the method comprising: obtaining a post-fermentation composition from a fermentation process used to produce recombinant egg protein, the post-fermentation composition comprising a cell culturing medium containing recombinant protein secreted by recombinant cells and recombinant cell byproducts; processing the post-fermentation composition under conditions that separate the recombinant protein from the plurality of recombinant cell byproducts without using a resin for separation, thereby obtaining a separated recombinant protein; concentrating the separated recombinant protein with a low fouling filtering system that filters the separated recombinant protein to produce a recombinant protein concentrate; and collecting the recombinant protein concentrate comprising recombinant egg protein for an ingredient composition in a consumable food product.

In some embodiments, the recombinant protein concentrate has a concentration of at least 400 g/L.

In one aspect, disclosed herein are methods for preparing an ingredient composition for a consumable food product comprising a recombinant protein concentrate, the method comprising: processing a cell culturing medium comprising: (i) recombinant cells that secrete a recombinant egg protein; (ii) the recombinant egg protein secreted; and (iii) recombinant cell byproducts, under conditions that separate the recombinant egg protein from the recombinant cell byproducts without using a resin, thereby obtaining a separated recombinant protein having a reduced quantity of the recombinant cell byproducts for preparation of the consumable food product; concentrating the separated recombinant protein with a low fouling filtering system that filters the separated recombinant protein to produce a recombinant protein concentrate; collecting the recombinant protein concentrate comprising recombinant egg protein; and preparing the ingredient composition comprising the recombinant protein concentrate for the consumable food product, the consumable food product comprising liquid egg, beverages, baked food, egg scrambles, dietary supplements, or gummy supplements.

In some embodiments, the egg protein comprises ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof. In some embodiments, the egg protein has a sequence that is at least 80% identical to the egg protein naturally produced in a bird, a chicken, quail, turkey, turkey vulture, hummingbird, duck, ostrich, goose, gull, guineafowl, pheasant, or emu.

In some embodiments, the consumable food product comprising the recombinant protein concentrate comprises liquid egg, beverages, baked food, dietary supplements, or gummy supplements.

In some embodiments, the recombinant protein concentrate provides an improvement to at least one additional feature selected from the group consisting of: flavor, moisture retention, water activity, mouthfeel, texture, hardness, cohesiveness, springiness, chewiness, stability to heat treatment, stability to pH, firmness, toughness, resilience, and stability for transportation.

In one aspect, disclosed herein are methods for preparing a consumable composition, the method comprising steps of: obtaining a composition comprising a recombinant protein and a plurality of recombinant cell byproducts, wherein the recombinant cell byproducts comprise an exopolysaccharide (EPS) and an off-flavor component, contacting, in a bioreactor, the composition with an anionic resin that reversibly attaches to the recombinant protein and does not substantially attach to the plurality of recombinant cell byproducts; separating the recombinant protein attached to the anionic resin from the plurality of recombinant cell byproducts; collecting the separated recombinant protein, thereby obtaining a protein product having a reduced quantity of the plurality of recombinant cell byproducts; and formulating a consumable composition comprising the protein product.

In some embodiments, wherein the composition comprising the recombinant protein and the plurality of recombinant cell byproducts is a cell culturing medium.

In accordance with any of the embodiments, the composition comprising the recombinant protein and the plurality of recombinant cell byproducts is a cell culture medium comprising recombinant cells that secreted the recombinant protein and the plurality of recombinant cell byproducts.

In accordance with any of the embodiments, the composition comprising the recombinant protein and the plurality of recombinant cell byproducts occurs in a bioreactor.

In accordance with any of the embodiments, the composition comprising the recombinant protein and the plurality of recombinant cell byproducts has a pH greater than the isoelectric point (pI) of the recombinant protein.

In accordance with any of the embodiments, the pH of the composition comprising the recombinant protein and the plurality of recombinant cell byproducts is not modified to achieve a pH greater than the pI of the recombinant protein.

In accordance with any of the embodiments, the anion resin is a strong anion exchange resin or a weak anion exchange resin.

In accordance with any of the embodiments, the anion resin is one or more of Capto Q resin, a DEAE type weak anion exchanger, a resin with trimethyl aminoethyl groups, a resin with triethyl aminoethyl groups, a resin with quaternary amine groups.

In accordance with any of the embodiments, the contacting step comprises addition of the anionic resin to a bioreactor comprising a cell culturing medium comprising recombinant cells that secrete the recombinant protein and the plurality of recombinant cell byproducts.

In accordance with any of the embodiments, the separating step comprises withdrawing the anionic resin from the bioreactor.

In accordance with any of the embodiments, the anionic resin is reintroduced into the bioreactor after separating the recombinant protein attached to the anionic resin.

In accordance with any of the embodiments, the separating step further comprises withdrawing the anionic resin an at least second time from the bioreactor.

In accordance with any of the embodiments, the anionic resin is reintroduced for an at least second time into the bioreactor after separating the recombinant protein attached to the anionic resin.

In accordance with any of the embodiments, the method further comprising a concentration step and/or diafiltration treatment of the separated recombinant protein to produce a protein-containing composition having a preferred pH and/or ionic condition.

In accordance with any of the embodiments, the protein-containing composition having a preferred pH and/or ionic condition is further heat treated and/or dried.

In accordance with any of the embodiments, the composition comprising the recombinant protein and the plurality of recombinant cell byproducts and/or the protein product having a reduced quantity of the plurality of recombinant cell byproducts is further heat treated and/or dried.

In accordance with any of the embodiments, the heat treatment separates the recombinant protein and the off-flavor component, wherein the heat is applied at a temperature and duration such that the off-flavor component is volatized and a gaseous off-flavor component is removable.

In accordance with any of the embodiments, a vacuum is applied contemporaneous with the application of heat and the vacuum facilitates removal of the gaseous off-flavor component.

In accordance with any of the embodiments, the off-flavor component is an acid, an alcohol, an aldehyde, an aromatic, an ester, or a ketone.

In accordance with any of the embodiments, the off-flavor component is (E)-2-nonenal; 1-dodecene; 1-hexanol, 2-ethyl-; 1-hexen-3-one; 1-octen-3-one; 2,3-butanedione; 2-butanone; 2-methylbutanal; 2-methylpropanal; 2-propanone; 2-undecanone; 3-methylbutanal; acetaldehyde; benzene ethanol; benzyl alcohol; butanal, 3-methyl-; chlorotoluene; nonanoic acid; p-cresol; or propanoic acid, 2-methyl-, 3-hydroxy-2,4,4-trimethylpentyl ester; benzaldehyde, phenylacetaldehyde; acetic acid; 2-methylpropanoic acid; butanoic acid; 2-methylbutanoic acid; 3-methylbutanoic acid; pentanoic acid; hexanoic acid.

In accordance with any of the embodiments, the temperature of the protein-containing composition having a preferred pH and/or ionic conditions, the composition comprising the recombinant protein and the plurality of recombinant cell byproducts, and/or the protein product having a reduced quantity of the plurality of recombinant cell byproducts during the heat treatment is up to 80° C., e.g., from about 50° C. to about 60° C.

In accordance with any of the embodiments, the method comprises agitation during the heat treatment.

In accordance with any of the embodiments, the heat treatment and/or drying step produces a dry protein product having a reduced quantity of the plurality of recombinant cell byproducts.

In accordance with any of the embodiments, the composition comprising the recombinant protein and the plurality of recombinant cell byproducts and/or the protein product having a reduced quantity of the plurality of recombinant cell byproducts further undergoes an oxidation step, e.g., comprising the addition of hydrogen peroxide.

In accordance with any of the embodiments, the ratio of the recombinant cell byproducts to recombinant protein in the composition comprising a recombinant protein and the plurality of recombinant cell byproducts is about 1:3 to about 3:1.

In accordance with any of the embodiments, the protein product has an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction an at least 75% reduction, at least 80% reduction, at least 90% reduction, or at least 95% reduction in the quantity of EPS and/or the quantity of off-flavor components relative to the composition comprising a recombinant protein and the plurality of recombinant cell byproducts.

In accordance with any of the embodiments, less than about 10% of the weight of the protein product comprises recombinant cell byproducts.

In accordance with any of the embodiments, less than about 5% of the weight of the protein product comprises recombinant cell byproducts.

In accordance with any of the embodiments, less than about 5%, less than about 1%, less than about 0.1%, or less than about 0.01% of the weight of the protein product comprises the off-flavor component.

In accordance with any of the embodiments, the off-flavor component in the protein product is virtually undetectable to a standard consumer.

In accordance with any of the embodiments, the EPS is generally inseparable from the recombinant protein when using size exclusion chromatography.

In accordance with any of the embodiments, the EPS is naturally a component of a recombinant cell's cell wall.

In accordance with any of the embodiments, the EPS has an apparent size of about 13 kDa to about 27 kDa as characterized by a size exclusion chromatography column.

In accordance with any of the embodiments, the EPS comprises mannose.

In accordance with any of the embodiments, the EPS further comprises N-acetylglucosamine and/or glucose.

In accordance with any of the embodiments, the EPS comprises about 91 mol % mannose, about 5 mol % N-acetylglucosamine, and about 3 mol % glucose as analyzed by gas chromatography in tandem with mass spectrometry.

In accordance with any of the embodiments, the EPS comprises an α(1,6)-linked backbone with α(1,2)-linked branches and/or α(1,3)-linked branches.

In accordance with any of the embodiments, the EPS is a mannan.

In accordance with any of the embodiments, the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

In accordance with any of the embodiments, the recombinant cell type is selected from Arxula spp., Arxula adeninivorans, Kluyveromyces spp., Kluyveromyces lactis, Komagataclla phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor michei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

In accordance with any of the embodiments, the fungus is a Pichia species.

In accordance with any of the embodiments, the Pichia species is Komagataella phaffii or Komagataella pastoris.

In accordance with any of the embodiments, the recombinant protein is an enzyme, a nutritive protein, a food ingredient, or a food additive.

In accordance with any of the embodiments, the enzyme is pepsinogen or pepsin.

In accordance with any of the embodiments, the protein is an egg-white protein.

In accordance with any of the embodiments, the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

In accordance with any of the embodiments, the egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

In accordance with any of the embodiments, the consumable composition comprising the protein product comprises food products, beverage products, or dietary supplements.

In accordance with any of the embodiments, the food products comprise baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings.

In accordance with any of the embodiments, the beverage products comprise soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks.

In accordance with any of the embodiments, the dietary supplements comprise multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement.

In accordance with any of the embodiments, the processing the composition under conditions that separate the recombinant protein from the plurality of recombinant cell byproducts further comprises one or more of: i) one or more cation exchange resin, rather than an anionic resin, that reversibly attach to the recombinant protein and does not substantially attach to the EPS, ii) an enzyme that digests the recombinant protein or the EPS, iii) an adsorbent that reversibly attaches to the EPS and does not substantially attach to the recombinant protein, iv) a flocculant that attaches to the EPS and does not substantially attach to the recombinant protein, v) an amphipathic solvent which separates the recombinant protein and the recombinant cell byproduct; vi) heat and vacuum conditions which separate the recombinant protein and the recombinant cell byproduct; and/or vii) oxidation.

In one aspect, disclosed herein are consumable compositions obtained by the method in accordance with any of the embodiments.

In one aspect, disclosed herein are methods for preparing a consumable composition, the method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises resuspending the dry protein composition in an amphipathic solvent, wherein the amphipathic solvent separates the recombinant protein from the recombinant cell byproduct; collecting the separated recombinant protein, thereby obtaining a protein product having a reduced quantity of the plurality of recombinant cell byproducts; and formulating a consumable composition comprising the protein product.

In some embodiments, the amphipathic solvent is an alcohol.

In accordance with any of the embodiments, the amphipathic solvent is ethanol.

In accordance with any of the embodiments, the method comprises agitation during the resuspending.

In accordance with any of the embodiments, the method further comprising a concentration step and/or diafiltration treatment of the separated recombinant protein to produce a protein-containing composition having a preferred pH and/or ionic condition.

In one aspect, disclosed herein are methods for preparing a consumable composition, the method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises one or more cation exchange resins that reversibly attach to the recombinant protein and do not substantially attach to the plurality of recombinant cell byproducts; collecting the separated recombinant protein, thereby obtaining a protein product having a reduced quantity of the plurality of recombinant cell byproducts; and formulating a consumable composition comprising the protein product.

In one aspect, disclosed herein are methods for preparing a consumable composition, the method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises an anionic resin that reversibly attach to the recombinant protein and do not substantially attach to the plurality of recombinant cell byproducts; collecting the separated recombinant protein, thereby obtaining a protein product having a reduced quantity of the plurality of recombinant cell byproducts; and formulating a consumable composition comprising the protein product.

In accordance with any of the embodiments, the dry protein composition is a spray dried protein powder.

In one aspect, disclosed herein are methods for preparing a consumable composition, the method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises resuspending the dry protein composition and a diafiltration treatment, an ultrafiltration step, and/or microfiltration step of the resuspended dry protein composition; collecting the separated recombinant protein, thereby obtaining a protein product having a reduced quantity of the plurality of recombinant cell byproducts; and formulating a consumable composition comprising the protein product.

In one aspect, disclosed herein are methods for preparing a consumable composition, the method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises an oxidation step (e.g., addition of hydrogen peroxide); collecting the separated recombinant protein, thereby obtaining a protein product having a reduced quantity of the plurality of recombinant cell byproducts; and formulating a consumable composition comprising the protein product.

In one aspect, disclosed herein are methods for preparing a consumable composition, the method comprising steps of: obtaining a dry protein composition comprising a recombinant protein and a plurality of recombinant cell byproducts, wherein the recombinant cell byproducts comprise an exopolysaccharide (EPS) and an off-flavor component; processing the dry protein composition under conditions that separate the recombinant protein from the plurality of recombinant cell byproducts, wherein the processing step comprises maintaining the composition under heat and vacuum conditions; collecting the separated recombinant protein, thereby obtaining a protein product having a reduced quantity of the plurality of recombinant cell byproducts; and formulating a consumable composition comprising the protein product.

Additionally, any composition or method disclosed herein is applicable to any herein-disclosed composition or method. In other words, any aspect or embodiment described herein can be combined with any other aspect or embodiment as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 to FIG. 10 are a flow diagrams illustrating method for producing a protein products or purified EPS of the present disclosure.

FIG. 11 is a chromatogram showing separation of recombinant proteins using a typical ion exchange resin listed above. Black line is the absorbance trace at 280 nm indicating the proteins.

FIG. 12 is a chromatogram of a process using SP 400 resin for purification of an illustrative protein, n=3, dotted line showing conductivity.

FIG. 13 is a chromatogram of a process using a combination of SP 400 and Sepragen S resin (2.75:1.25 ratio) for purification of an illustrative protein, n=3, dotted line showing conductivity.

FIG. 14A is a chromatogram of the fractions in a process using anion exchange Capto Q resin for purification of an illustrative protein. The curved line with the first two rounded peaks showing protein elution (280 nm), the angular line shows the elution buffer B used in the process and the curve shows the usage during the process, the curve with the final peak shows conductivity (as, expected conductivity is high during protein elution and very high during CIP elution).

FIG. 14B is an SDS-PAGE gel of the fractions shown in FIG. 14A. Lane 2 is the feed, lane 3 is the flow through, lane 4 is the elute, and lane 5 is the cleaning in place (CIP).

FIG. 15 is a chart showing the absorption spectra of the supernatants from the study in Example 4. The higher pH showed higher absorbance across the board indicating protein is more stable at pH 6 than pH 4. But at pH 4.0 across the various adsorbents tested, bentonite BE125 showed the most decrease in the absorbance. At 350 nm, from top to bottom, the curves are Relisorb SP400; No filter aid, pH4; superimposed EZ DE—Celite 545—No filter aid, pH6; superimposed DIAON HPA25L—Chitosan 85% deacetylated; and Bentonite BE125.

FIG. 16 is a graph showing EPS analysis of the supernatant generated from Example 4.

FIG. 17 is a graph showing protein of interest analysis of the supernatants from Example 4.

FIG. 18 compares controls and various reprocessing methods. A curve surrounding the central core of the pentagon is the most neutral and preferred protein product. For data relating to taste (pointing towards the right of the pentagon), the worst performer is the control; then heat and vacuum; then IEX, heat, and vacuum; then ethanol; and the best performer was ion exchange. For data relating to appearance (at the top), the worst performer was the heat and vacuum; then control; then IEX, heat, and vacuum; then ion exchange; and the best performer was ethanol.

FIG. 19 illustrates an exemplary method for preparing purified recombinant protein from dried recombinant protein in accordance with some embodiments described.

FIG. 20 illustrates an exemplary method for preparing purified recombinant protein from dried recombinant protein in accordance with some embodiments described.

FIG. 21 compares performance of typical ultrafiltration membrane and low fouling filtration membrane.

FIG. 22 illustrates an exemplary method for preparing purified recombinant protein from a post-fermentation composition in accordance with some embodiments described.

FIG. 23 illustrates an exemplary method for preparing purified recombinant protein from a post-fermentation composition in accordance with some embodiments described.

### DETAILED DESCRIPTION

The present disclosure provides methods for producing consumable recombinant proteins that are substantially free from herein-disclosed undesired byproducts.

It has been discovered that when recombinant proteins are produced by fermenting yeast cells, such as Pichia, the recombinant cells likewise produces recombinant cell byproducts. The recombinant cell byproduct component may be produced in an about equal proportion as the recombinant protein, e.g., when the recombinant cell byproduct is an exopolysaccharide (EPS). This results in lower concentration of the recombinant protein in a resulting protein product or consumable composition. In some cases, the presence of recombinant cell byproducts in a protein product or consumable composition may have a non-preferred taste. Moreover, the presence of recombinant cell byproduct in a protein product or consumable composition will have different properties, such as density, viscosity, gelling, and flavor, relative to a protein product or consumable composition that lacks the recombinant cell byproduct. Accordingly, methods for processing a composition comprising a recombinant protein and a recombinant cell byproduct, e.g., EPS and/or an off-flavor component to separate the recombinant protein and the recombinant cell byproduct is needed.

### Resin-Based Purification

An aspect of the present disclosure is a method for preparing a protein product having a reduced quantity of a recombinant cell byproduct. The method comprises steps of: obtaining a composition comprising a recombinant protein and a recombinant cell byproduct; processing the composition under conditions that separate the recombinant protein and the recombinant cell byproduct, wherein the processing comprises a resin that reversibly attaches to the recombinant protein and does not substantially attach to the recombinant cell byproduct; and collecting the separated recombinant protein, thereby obtaining a protein product having a reduced quantity of the recombinant cell byproduct. In this method, the recombinant cell byproduct is an exopolysaccharide (EPS) or an off-flavor component.

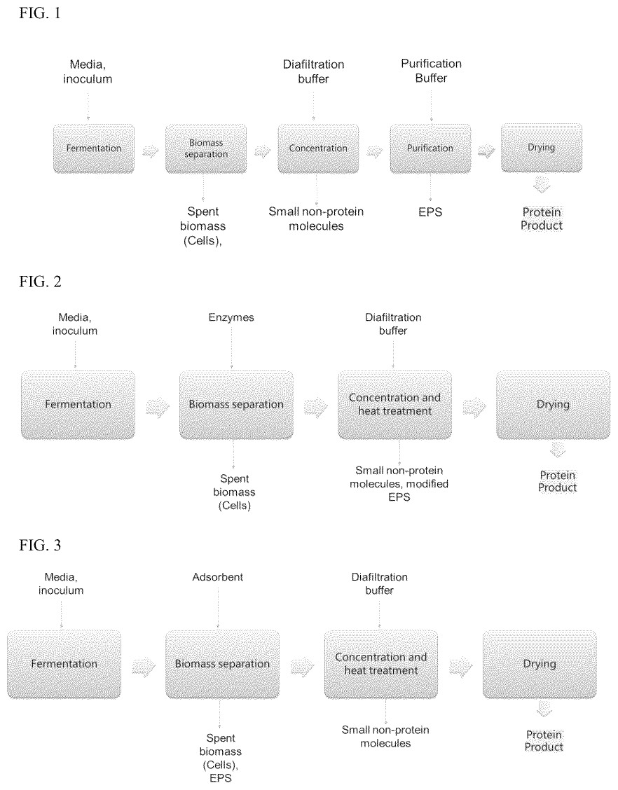

A illustrative method for producing a composition comprising a recombinant protein and a recombinant cell byproduct and separate the recombinant protein and the recombinant cell byproduct is shown in FIG. 1. The method of this aspect can be used to separate any extracellular product that is produced during a fermentation processes. The recombinant cell byproduct (e.g., EPS or off-flavor component) molecules are not ionic in nature and will flow through in an ion exchange column.

In a variation of the process shown in FIG. 1, the biomass separation step is omitted, and an anionic resin that reversibly attaches to the recombinant protein and does not substantially attach to the recombinant cell byproduct is added to the bioreactor. In some aspects, the method comprises withdrawing the resin from the bioreactor that has floated to the top of the culture and separating the recombinant protein attached to the anionic resin. In further aspects, the method further comprises sterilizing the resin (e.g., by autoclave) and reintroducing the resin into the reactor to repeat the process.

In embodiments, the resin is an anion exchanger or the resin is a cation exchange resin. In some cases, the cation exchanger is a strong cation exchange resin or a weak cation exchange resin. In some embodiments, the strong cation exchange resin is a sulphonate-type resin or the weak cation exchange resin is a carboxymethyl-type resin.

Any commercially-available resin that is capable of binding protein may be used.

An aspect of the present disclosure is a method for preparing a consumable composition. The method comprising the steps of: obtaining a composition comprising a recombinant protein and a recombinant cell byproduct; contacting the composition with an anionic resin that reversibly attaches to the recombinant protein and does not substantially attach to the plurality of recombinant cell byproducts; and collecting the separated recombinant protein, thereby obtaining a protein product having a reduced quantity of the recombinant cell byproduct.

In embodiments, wherein the composition comprising the recombinant protein and the plurality of recombinant cell byproducts is a cell culturing medium.

In some embodiments, wherein the composition comprising the recombinant protein and the plurality of recombinant cell byproducts is a cell culture medium comprising recombinant cells that secreted the recombinant protein and the plurality of recombinant cell byproducts.

In some embodiments, wherein the composition comprising the recombinant protein and the plurality of recombinant cell byproducts occurs in a bioreactor.

In some embodiments, the composition comprising the recombinant protein and the plurality of recombinant cell byproducts has a pH greater than the isoelectric point (pI) of the recombinant protein.

In some embodiments, wherein the pH of the composition comprising the recombinant protein and the plurality of recombinant cell byproducts is not modified to achieve a pH greater than the pI of the recombinant protein.

In some embodiments, the anion resin is a strong anion exchange resin or a weak anion exchange resin.

In some embodiments, wherein the anion resin is one or more of Capto Q resin, a DEAE type weak anion exchanger, a resin with trimethyl aminoethyl groups, a resin with triethyl aminoethyl groups, a resin with quaternary amine groups.

In some embodiments, the contacting step comprises addition of the anionic resin to a bioreactor comprising a cell culturing medium comprising recombinant cells that secrete the recombinant protein and the plurality of recombinant cell byproducts.

In some embodiments, the separating step comprises withdrawing the anionic resin from the bioreactor.

In some embodiments, the anionic resin is reintroduced into the bioreactor after separating the recombinant protein attached to the anionic resin.

In some embodiments, the separating step further comprises withdrawing the anionic resin an at least second time from the bioreactor.

In some embodiments, the anionic resin is reintroduced for an at least second time into the bioreactor after separating the recombinant protein attached to the anionic resin.

In various embodiments, the method further comprises a concentration step and/or diafiltration treatment of the separated recombinant protein to produce a protein-containing composition having a preferred pH and/or ionic condition.

In some embodiments, the protein-containing composition having a preferred pH and/or ionic condition is further heat treated and/or dried.

In some embodiments, the composition comprising the recombinant protein and the plurality of recombinant cell byproducts and/or the protein product having a reduced quantity of the plurality of recombinant cell byproducts is further heat treated and/or dried.

In some embodiments, the heat treatment separates the recombinant protein and the off-flavor component, wherein the heat is applied at a temperature and duration such that the off-flavor component is volatized and a gaseous off-flavor component is removable.

In some embodiments, a vacuum is applied contemporaneous with the application of heat and the vacuum facilitates removal of the gaseous off-flavor component.

In some embodiments, wherein the off-flavor component is an acid, an alcohol, an aldehyde, an aromatic, an ester, or a ketone.

In some embodiments, the off-flavor component is (E)-2-nonenal; 1-dodecene; 1-hexanol, 2-ethyl-; 1-hexen-3-one; 1-octen-3-one; 2,3-butanedione; 2-butanone; 2-methylbutanal; 2-methylpropanal; 2-propanone; 2-undecanone; 3-methylbutanal; acetaldehyde; benzene ethanol; benzyl alcohol; butanal, 3-methyl-; chlorotoluene; nonanoic acid; p-cresol; or propanoic acid, 2-methyl-, 3-hydroxy-2,4,4-trimethylpentyl ester; benzaldehyde, phenylacetaldehyde; acetic acid; 2-methylpropanoic acid; butanoic acid; 2-methylbutanoic acid; 3-methylbutanoic acid; pentanoic acid; hexanoic acid.

In some embodiments, the temperature of the protein-containing composition having a preferred pH and/or ionic conditions, the composition comprising the recombinant protein and the plurality of recombinant cell byproducts, and/or the protein product having a reduced quantity of the plurality of recombinant cell byproducts during the heat treatment is up to 80° C., e.g., from about 50° C. to about 60° C.

In some embodiments, the method comprises agitation during the heat treatment.

In some embodiments, the heat treatment and/or drying step produces a dry protein product having a reduced quantity of the plurality of recombinant cell byproducts.

In some embodiments, the composition comprising the recombinant protein and the plurality of recombinant cell byproducts and/or the protein product having a reduced quantity of the plurality of recombinant cell byproducts further undergoes an oxidation step, e.g., comprising the addition of hydrogen peroxide.

In some embodiments, the ratio of the recombinant cell byproducts to recombinant protein in the composition comprising a recombinant protein and the plurality of recombinant cell byproducts is about 1:3 to about 3:1.

In some embodiments, the protein product has an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction an at least 75% reduction, at least 80% reduction, at least 90% reduction, or at least 95% reduction in the quantity of EPS and/or the quantity of off-flavor components relative to the composition comprising a recombinant protein and the plurality of recombinant cell byproducts.

In some embodiments, less than about 10% of the weight of the protein product comprises recombinant cell byproducts.

In some embodiments, less than about 5% of the weight of the protein product comprises recombinant cell byproducts.

In some embodiments, less than about 5%, less than about 1%, less than about 0.1%, or less than about 0.01% of the weight of the protein product comprises the off-flavor component.

In some embodiments, wherein the off-flavor component in the protein product is virtually undetectable to a standard consumer.

In some embodiments, the EPS is generally inseparable from the recombinant protein when using size exclusion chromatography.

In some embodiments, the EPS is naturally a component of a recombinant cell's cell wall.

In some embodiments, the EPS has an apparent size of about 13 kDa to about 27 kDa as characterized by a size exclusion chromatography column.

In some embodiments, the EPS comprises mannose.

In some embodiments, the EPS further comprises N-acetylglucosamine and/or glucose.

In some embodiments, wherein the EPS comprises about 91 mol % mannose, about 5 mol % N-acetylglucosamine, and about 3 mol % glucose as analyzed by gas chromatography in tandem with mass spectrometry.

In some embodiments, the EPS comprises an α(1,6)-linked backbone with α(1,2)-linked branches and/or α(1,3)-linked branches.

In some embodiments, the EPS is a mannan.

In some embodiments, the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

In some embodiments, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor michei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

In some embodiments, the fungus is a Pichia species.

In some embodiments, the Pichia species is Komagataella phaffii or Komagataella pastoris.

In some embodiments, the recombinant protein is an enzyme, a nutritive protein, a food ingredient, or a food additive

In some embodiments, the enzyme is pepsinogen or pepsin.

In some embodiments, the protein is an egg-white protein.

In some embodiments, the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

In some embodiments, the egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

In some embodiments, the consumable composition comprising the protein product comprises food products, beverage products, or dietary supplements.

In some embodiments, the food products comprise baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings.

In some embodiments, the beverage products comprise soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks.

In some embodiments, wherein the dietary supplements comprise multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement.

In some embodiments, wherein the processing the composition under conditions that separate the recombinant protein from the plurality of recombinant cell byproducts further comprises one or more of: i) one or more cation exchange resin, rather than an anionic resin, that reversibly attach to the recombinant protein and does not substantially attach to the EPS, ii) an enzyme that digests the recombinant protein or the EPS, iii) an adsorbent that reversibly attaches to the EPS and does not substantially attach to the recombinant protein, iv) a flocculant that attaches to the EPS and does not substantially attach to the recombinant protein, v) an amphipathic solvent which separates the recombinant protein and the recombinant cell byproduct; vi) heat and vacuum conditions which separate the recombinant protein and the recombinant cell byproduct; and/or vii) oxidation.

In another aspect, the present disclosure provides a consumable composition obtained by any above-disclosed method.

An aspect of the present disclosure is a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the composition under conditions that separate the recombinant protein from the plurality of recombinant cell byproducts, wherein the processing step comprises an anionic resin that reversibly attaches to the recombinant protein and does not substantially attach to the plurality of recombinant cell byproducts; collecting the separated recombinant protein, thereby obtaining a protein product having a reduced quantity of the plurality of recombinant cell byproducts; and formulating a consumable composition comprising the protein product. In some embodiments, the dry protein composition is a spray dried protein powder.

In some embodiments, wherein the anion resin is a strong anion exchange resin or a weak anion exchange resin.

In various embodiments, wherein the anion resin is one or more of Capto Q resin, a DEAE type weak anion exchanger, a resin with trimethyl aminoethyl groups, a resin with triethyl aminoethyl groups, a resin with quaternary amine groups.

In several embodiments, wherein the anion resin is a component of a chromatography system.

In embodiments, wherein the chromatography system operates in batch mode with an axial flow column or a radial flow column or a centrifugal column or by use of a membrane chromatography column.

In some embodiments, wherein the chromatography system operates in a continuous mode comprising multiple columns in parallel, with the feed to the columns being switchable such that various steps in a chromatography process (e.g., equilibration, load, elute, and clean), occur contemporaneously.

In various embodiments, wherein the continuous mode comprises a simulated moving bed (SMB) or an Ion Separator (e.g., ISEP®) system.

In several embodiments, wherein the dry protein composition comprising the recombinant protein and the plurality of recombinant cell byproducts was previously treated to remove spent biomass including recombinant cells and/or was previously treated to remove small non-protein molecules.

In embodiments, wherein the treatment to remove small non-protein molecules comprises a step that concentrates the dry protein composition comprising the recombinant protein and the plurality of recombinant cell byproducts.

In some embodiments, wherein the treatment to remove small non-protein molecules comprises a diafiltration buffer.

In various embodiments, the method further comprises a concentration step and/or diafiltration treatment of the separated recombinant protein to produce a protein-containing composition having a preferred pH and/or ionic condition.

In several embodiments, wherein the protein-containing composition having a preferred pH and/or ionic condition is further heat treated and/or dried.

In embodiments, wherein the dry protein composition comprising the recombinant protein and the plurality of recombinant cell byproducts and/or the protein product having a reduced quantity of the plurality of recombinant cell byproducts is further heat treated and/or dried.

In some embodiments, wherein the heat treatment separates the recombinant protein and the off-flavor component, wherein the heat is applied at a temperature and duration such that the off-flavor component is volatized and a gaseous off-flavor component is removable.

In various embodiments, wherein a vacuum is applied contemporaneous with the application of heat and the vacuum facilitates removal of the gaseous off-flavor component.

In several embodiments, wherein the off-flavor component is an acid, an alcohol, an aldehyde, an aromatic, an ester, or a ketone.

In embodiments, wherein the off-flavor component is (E)-2-nonenal; 1-dodecene; 1-hexanol, 2-ethyl-; 1-hexen-3-one; 1-octen-3-one; 2,3-butanedione; 2-butanone; 2-methylbutanal; 2-methylpropanal; 2-propanone; 2-undecanone; 3-methylbutanal; acetaldehyde; benzene ethanol; benzyl alcohol; butanal, 3-methyl-; chlorotoluene; nonanoic acid; p-cresol; or propanoic acid, 2-methyl-, 3-hydroxy-2,4,4-trimethylpentyl ester; benzaldehyde, phenylacetaldehyde; acetic acid; 2-methylpropanoic acid; butanoic acid; 2-methylbutanoic acid; 3-methylbutanoic acid; pentanoic acid; hexanoic acid.

In some embodiments, wherein the temperature of the protein-containing composition having a preferred pH and/or ionic conditions, the dry protein composition comprising the recombinant protein and the plurality of recombinant cell byproducts, and/or the protein product having a reduced quantity of the plurality of recombinant cell byproducts during the heat treatment is up to 80° C., e.g., from about 50° C. to about 60° C.

In various embodiments, wherein the method comprises agitation during the heat treatment.

In several embodiments, wherein the heat treatment and/or drying step produces a dry protein product having a reduced quantity of the plurality of recombinant cell byproducts.

In embodiments, wherein the dry protein composition comprising the recombinant protein and the plurality of recombinant cell byproducts and/or the protein product having a reduced quantity of the plurality of recombinant cell byproducts further undergoes an oxidation step, e.g., comprising the addition of hydrogen peroxide.

In some embodiments, wherein the ratio of the recombinant cell byproducts to recombinant protein in the dry protein composition comprising a recombinant protein and the plurality of recombinant cell byproducts is about 1:3 to about 3:1.

In various embodiments, wherein the protein product has an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction an at least 75% reduction, at least 80% reduction, at least 90% reduction, or at least 95% reduction in the quantity of EPS and/or the quantity of off-flavor components relative to the dry protein composition comprising a recombinant protein and the plurality of recombinant cell byproducts.

In several embodiments, wherein less than about 10% of the weight of the protein product comprises recombinant cell byproducts.

In embodiments, wherein less than about 5% of the weight of the protein product comprises recombinant cell byproducts.

In some embodiments, wherein less than about 5%, less than about 1%, less than about 0.1%, or less than about 0.01% of the weight of the protein product comprises the off-flavor component.

In various embodiments, wherein the off-flavor component in the protein product is virtually undetectable to a standard consumer.

In several embodiments, wherein the EPS is generally inseparable from the recombinant protein when using size exclusion chromatography.

In embodiments, wherein the EPS is naturally a component of a recombinant cell's cell wall.

In some embodiments, wherein the EPS has an apparent size of about 13 kDa to about 27 kDa as characterized by a size exclusion chromatography column.

In various embodiments, wherein the EPS comprises mannose.

In several embodiments, wherein the EPS further comprises N-acetylglucosamine and/or glucose.

In embodiments, wherein the EPS comprises about 91 mol % mannose, about 5 mol % N-acetylglucosamine, and about 3 mol % glucose as analyzed by gas chromatography in tandem with mass spectrometry.

In some embodiments, wherein the EPS comprises an α(1,6)-linked backbone with α(1,2)-linked branches and/or α(1,3)-linked branches.

In various embodiments, wherein the EPS is a mannan.

In several embodiments, wherein the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

In embodiments, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor michei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

In some embodiments, wherein the fungus is a Pichia species.

In various embodiments, wherein the Pichia species is Komagataella phaffii or Komagataclla pastoris.

In several embodiments, wherein the recombinant protein is an enzyme, a nutritive protein, a food ingredient, or a food additive.

In embodiments, wherein the enzyme is pepsinogen or pepsin.

In some embodiments, wherein the protein is an egg-white protein.

In various embodiments, wherein the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

In several embodiments, wherein the egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

In embodiments, wherein the consumable composition comprising the protein product comprises food products, beverage products, or dietary supplements.

In some embodiments, wherein the food products comprise baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings.

In various embodiments, wherein the beverage products comprise soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks.

In several embodiments, wherein the dietary supplements comprise multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement.

In embodiments, wherein the processing the composition under conditions that separate the recombinant protein from the plurality of recombinant cell byproducts further comprises one or more of: i) one or more cation exchange resin, rather than an anionic resin that reversibly attach to the recombinant protein and does not substantially attach to the EPS, ii) an enzyme that digests the recombinant protein or the EPS, iii) an adsorbent that reversibly attaches to the EPS and does not substantially attach to the recombinant protein, and/or iv) a flocculant that attaches to the EPS and does not substantially attach to the recombinant protein.

In another aspect, the present disclosure provides a consumable composition obtained by any above-disclosed method.

Another aspect of the present disclosure is a method for preparing a consumable composition. The method comprising steps of: obtaining a dry protein composition comprising a recombinant protein and a plurality of recombinant cell byproducts, wherein the recombinant cell byproducts comprise an exopolysaccharide (EPS) and an off-flavor component; processing the composition under conditions that separate the recombinant protein from the plurality of recombinant cell byproducts, wherein the processing step comprises one or more cation exchange resins that reversibly attach to the recombinant protein and do not substantially attach to the plurality of recombinant cell byproducts; collecting the separated recombinant protein, thereby obtaining a protein product having a reduced quantity of the plurality of recombinant cell byproducts; and formulating a consumable composition comprising the protein product.

In some embodiments, wherein the one or more cation exchange resins comprise a strong cation exchange resin, e.g., a sulfopropyl-, sulfomethyl-, or sulphonate-type resin, and/or a weak cation exchange resin, e.g., a carboxymethyl-type resin.

In various embodiments, wherein the one or more cation exchange resins comprise poly styrene divinyl benzene, poly methacrylate or cellulose or cross-linked dextran or cross-linked agarose or inorganic materials coated with hydrophilic polymers.

In several embodiments, wherein the one or more cation exchange resins have a particle size of from about 50 μm and about 200 μm and/or have a protein binding capacity of from about 50 to about 100 g protein/L resin.

In embodiments, wherein the one or more cation exchange resins comprise Cytiva Capto S, HP20, resindion SP400, Sepragen S, SP20, and/or Mitsubishi Relisorb EXE349.

In some embodiments, wherein the processing step comprises two cationic resins, wherein the two cationic resins are in a ratio of 1:5 to 5:1, 1:4 to 4:1, 1:3 to 3:1, 1:2 to 2:1, or 1:1.

In various embodiments, wherein the two resins are SP400 and Sepragen S and in a ratio of about 3:1, e.g., 2.75:1.25.

In several embodiments, wherein the dry protein composition comprising the recombinant protein and the plurality of recombinant cell byproducts lacks recombinant cells that secreted the recombinant protein and the plurality of recombinant cell byproducts.

In embodiments, wherein the dry protein composition comprising the recombinant protein and the plurality of recombinant cell byproducts has a pH less than the isoelectric point (pI) of the recombinant protein, which is achieved by lowering the pH of the composition comprising the recombinant protein and the plurality of recombinant cell byproducts.

In some embodiments, wherein the one or more cationic resins are components of a chromatography system, wherein the chromatography system operates in batch mode with an axial flow column or a radial flow column or a centrifugal column or by use of a membrane chromatography column.

In various embodiments, wherein the one or more cationic resins are components of a chromatography system, wherein the chromatography system operates in a continuous mode comprising multiple columns in parallel, with the feed to the columns being switchable such that various steps in a chromatography process (e.g., equilibration, load, elute, and clean), occur contemporaneously.

In several embodiments, wherein the continuous mode comprises a simulated moving bed (SMB) or an Ion Separator (e.g., ISEP®) system.

In embodiments, wherein the dry protein composition comprising the recombinant protein and the plurality of recombinant cell byproducts was previously treated to remove spent biomass including recombinant cells and/or was previously treated to remove small non-protein molecules.

In some embodiments, wherein the treatment to remove small non-protein molecules comprises a step that concentrates the composition comprising the recombinant protein and the plurality of recombinant cell byproducts.

In various embodiments, wherein the treatment to remove small non-protein molecules comprises a diafiltration buffer.

In several embodiments, the method further comprises a concentration step and/or diafiltration treatment of the separated recombinant protein to produce a protein-containing composition having a preferred pH and/or ionic condition.

In embodiments, wherein the protein-containing composition having a preferred pH and/or ionic condition is further heat treated and/or dried.

In some embodiments, wherein the dry protein composition comprising the recombinant protein and the plurality of recombinant cell byproducts and/or the protein product having a reduced quantity of the plurality of recombinant cell byproducts is further heat treated and/or dried.

In various embodiments, wherein the heat treatment separates the recombinant protein and the off-flavor component, wherein the heat is applied at a temperature and duration such that the off-flavor component is volatized and a gaseous off-flavor component is removable.

In several embodiments, wherein a vacuum is applied contemporaneous with the application of heat and the vacuum facilitates removal of the gaseous off-flavor component.

In embodiments, wherein the off-flavor component is an acid, an alcohol, an aldehyde, an aromatic, an ester, or a ketone.

In some embodiments, wherein the off-flavor component is (E)-2-nonenal; 1-dodecene; 1-hexanol, 2-ethyl-; 1-hexen-3-one; 1-octen-3-one; 2,3-butanedione; 2-butanone; 2-methylbutanal; 2-methylpropanal; 2-propanone; 2-undecanone; 3-methylbutanal; acetaldehyde; benzene ethanol; benzyl alcohol; butanal, 3-methyl-; chlorotoluene; nonanoic acid; p-cresol; or propanoic acid, 2-methyl-, 3-hydroxy-2,4,4-trimethylpentyl ester; benzaldehyde, phenylacetaldehyde; acetic acid; 2-methylpropanoic acid; butanoic acid; 2-methylbutanoic acid; 3-methylbutanoic acid; pentanoic acid; hexanoic acid.

In various embodiments, wherein the temperature of the protein-containing composition having a preferred pH and/or ionic conditions, the dry protein composition comprising the recombinant protein and the plurality of recombinant cell byproducts, and/or the protein product having a reduced quantity of the plurality of recombinant cell byproducts during the heat treatment is up to 80° C., e.g., from about 50° C. to about 60° C.

In several embodiments, wherein the method comprises agitation during the heat treatment.

In embodiments, wherein the heat treatment and/or drying step produces a dry protein product having a reduced quantity of the plurality of recombinant cell byproducts.

In some embodiments, wherein the dry protein composition comprising the recombinant protein and the plurality of recombinant cell byproducts and/or the protein product having a reduced quantity of the plurality of recombinant cell byproducts further undergoes an oxidation step, e.g., comprising the addition of hydrogen peroxide.

In various embodiments, wherein the ratio of the recombinant cell byproducts to recombinant protein in the dry protein composition comprising a recombinant protein and the plurality of recombinant cell byproducts is about 1:3 to about 3:1.

In several embodiments, wherein the protein product has an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction an at least 75% reduction, at least 80% reduction, at least 90% reduction, or at least 95% reduction in the quantity of EPS and/or the quantity of off-flavor components relative to the dry protein composition comprising a recombinant protein and the plurality of recombinant cell byproducts.

In embodiments, wherein less than about 10% of the weight of the protein product comprises recombinant cell byproducts.

In some embodiments, wherein less than about 5% of the weight of the protein product comprises recombinant cell byproducts.

In various embodiments, wherein less than about 5%, less than about 1%, less than about 0.1%, or less than about 0.01% of the weight of the protein product comprises the off-flavor component.

In several embodiments, wherein the off-flavor component in the protein product is virtually undetectable to a standard consumer.

In embodiments, wherein the EPS is generally inseparable from the recombinant protein when using size exclusion chromatography.

In some embodiments, wherein the EPS is naturally a component of a recombinant cell's cell wall.

In various embodiments, wherein the EPS has an apparent size of about 13 kDa to about 27 kDa as characterized by a size exclusion chromatography column.

In several embodiments, wherein the EPS comprises mannose.

In embodiments, wherein the EPS further comprises N-acetylglucosamine and/or glucose.

In some embodiments, wherein the EPS comprises about 91 mol % mannose, about 5 mol % N-acetylglucosamine, and about 3 mol % glucose as analyzed by gas chromatography in tandem with mass spectrometry.

In various embodiments, wherein the EPS comprises an α(1,6)-linked backbone with α(1,2)-linked branches and/or α(1,3)-linked branches.

In several embodiments, wherein the EPS is a mannan.

In embodiments, wherein the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

In some embodiments, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor michei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

In various embodiments, wherein the fungus is a Pichia species.

In several embodiments, wherein the Pichia species is Komagataella phaffii or Komagataella pastoris.

In embodiments, wherein the recombinant protein is an enzyme, a nutritive protein, a food ingredient, or a food additive.

In some embodiments, wherein the enzyme is pepsinogen or pepsin.

In various embodiments, wherein the protein is an egg-white protein.

In several embodiments, wherein the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

In embodiments, wherein the egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

In some embodiments, wherein the consumable composition comprising the protein product comprises food products, beverage products, or dietary supplements.

In various embodiments, wherein the food products comprise baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings.

In several embodiments, wherein the beverage products comprise soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks.

In embodiments, wherein the dietary supplements comprise multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement.

In some embodiments, wherein the processing the composition under conditions that separate the recombinant protein from the plurality of recombinant cell byproducts further comprises one or more of: i) an anionic resin that reversibly attaches to the recombinant protein and does not substantially attach to the EPS, ii) an enzyme that digests the recombinant protein or the EPS, iii) an adsorbent that reversibly attaches to the EPS and does not substantially attach to the recombinant protein, and/or iv) a flocculant that attaches to the EPS and does not substantially attach to the recombinant protein.

In another aspect, the present disclosure provides a consumable composition obtained by any above-disclosed method.

In various embodiments, the resin is a component of a chromatography system. In some cases, the chromatography system operates in batch mode with an axial flow column or a radial flow column or a centrifugal column or by use of a membrane chromatography column or the chromatography system operates in a continuous mode comprising multiple columns in parallel, with the feed to the columns being switchable such that various steps in a chromatography process (e.g., equilibration, load, elute, and clean), occur contemporaneously. In various cases, the continuous mode comprises a simulated moving bed (SMB) or an Ion Separator (e.g., ISEP®) system.

In various embodiments, the resin is not a component of a chromatography system. In such embodiments, the resin is pumped into a bioreactor comprising a recombinant protein and a recombinant cell byproduct. In some instances, the composition comprising a recombinant protein and a recombinant cell byproduct is a cell culture medium comprising recombinant cells that secreted the recombinant protein and the plurality of recombinant cell byproducts. In some cases, the resin floats to the top of the culture and is withdrawn from the bioreactor.

In embodiments, the composition comprising a recombinant protein and a recombinant cell byproduct was previously treated to remove spent biomass including recombinant cells and was previously treated to remove small non-protein molecules. In some cases, the treatment to remove small non-protein molecules comprises a step that concentrates the composition comprising the recombinant protein and the recombinant cell byproduct. In embodiments, the treatment to remove small non-protein molecules comprises a diafiltration buffer.

In some embodiments, the method further comprises a concentration step and/or diafiltration treatment of the separated recombinant protein to produce a protein-containing composition having a preferred pH and/or ionic conditions. In embodiments, protein-containing composition having a preferred pH and/or ionic conditions is further heat treated and/or dried.

An illustrative chromatogram showing the purification of a recombinant protein using a cation exchange column SP400 is shown in FIG. 11. The recombinant proteins are bound to the column eluted in Elution Zones 1 and 2 (beginning at 8 minutes and 11 minutes, respectively). More specifically, the fraction in Elution Zone 2 is the recombinant protein of interest separated from the unbound peak during loading (around 5 minutes), bound product peak during clean-in-place (CIP; around 16 minutes), and the other loosely bound proteins in Elution Zone 1 (around 8 minutes).

In some embodiments, a variation of the process shown in FIG. 1 would be to equilibrate the column in the elution buffer 1 pre feed application to elute the host cell proteins along with the recombinant cell byproduct impurities, e.g., EPS or off-flavor component.

In another variation of the process shown in FIG. 1, the concentration step is omitted, and a microfiltered fermentation supernatant is loaded onto the column that is equilibrated in elution 1 buffer; thereby separating the recombinant cell byproduct (e.g., EPS or off-flavor component), small molecule impurities and the host cell proteins in the column loading step.

Processes for protein separation and intracellular protein separation have been described in the literature, see, e.g., U.S. Pat. Nos. 10,857,483 and 9,821,249; the contents of each of which is incorporated herein by reference in its entirety.

### Hydrophobic Solvent or Amphipathic Solvent-Based Purification

An aspect of the present disclosure is a method for preparing a protein product having a reduced quantity of a recombinant cell byproduct. The method comprises steps of: obtaining a dry protein composition comprising a recombinant protein and a recombinant cell byproduct; processing the dry protein composition under conditions that separate the recombinant protein from the plurality of recombinant cell byproducts, wherein the processing step comprises resuspending the dry protein composition in an amphipathic solvent, wherein the amphipathic solvent separates the recombinant protein from the recombinant cell byproduct; collecting the separated recombinant protein, thereby obtaining a protein product having a reduced quantity of the recombinant cell byproduct. In some instances, the recombinant cell byproduct is an off-flavor component.

In some embodiments, the amphipathic solvent is an alcohol.

In some embodiments, the amphipathic solvent is ethanol. In some embodiments, the ethanol is in a 10% v/v solution.

In some embodiments, the step of processing the composition comprises resuspending the dry protein composition in the amphipathic solvent (e.g., ethanol). In some embodiments, the processing step comprises resuspending the dry protein composition to a concentration of about 50 g/L.

In some embodiments, the method further comprises agitation during the resuspending.

A illustrative method for producing a composition comprising a recombinant protein and a recombinant cell byproduct and separate the recombinant protein and the recombinant cell byproduct is shown in FIG. 6.

In embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In various embodiments, the composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic conditions. In some cases, protein-containing composition having a preferred pH and/or ionic conditions is further heat treated and/or dried. The heat treatment and/or drying step may produce a dry protein product having a reduced quantity of the off-flavor component.

In various embodiments, the protein product having a reduced quantity of the off-flavor component comprises an at least 50% reduction in off-flavor component quantity relative to the composition comprising a recombinant protein and a recombinant cell byproduct. In some cases, the protein product has an at least 75% reduction, at least 80% reduction, at least 90% reduction, or at least 95% reduction in the off-flavor component relative to the composition comprising a recombinant protein and a recombinant cell byproduct.

In embodiments, less than about 5%, less than about 1%, less than about 0.1%, or less than about 0.01% of the weight of the protein product comprises the off-flavor component. In some cases, the off-flavor component in the protein product is virtually undetectable to a standard consumer.

### Oxidation-Based Purification

An aspect of the present disclosure is a method for preparing a protein product having a reduced quantity of a recombinant cell byproduct. The method comprises steps of: obtaining a dry protein composition comprising a recombinant protein and a recombinant cell byproduct; processing the dry protein composition under conditions that separate the recombinant protein from the plurality of recombinant cell byproducts, wherein the processing step comprises an oxidation step; collecting the separated recombinant protein, thereby obtaining a protein product having a reduced quantity of the recombinant cell byproduct. In some instances, the recombinant cell byproduct is an off-flavor component.

In some embodiments, the oxidation step comprises addition of hydrogen peroxide. In some embodiments, hydrogen peroxide is added to the composition such that the final solution is 3% v/v hydrogen peroxide.

In some embodiments, prior to addition of the hydrogen peroxide, the pH is adjusted to a pH of 4. In some embodiments, the pH is adjusted using phosphoric acid.

In some embodiments, the processing step further comprises mixing the solution for about 6 hours.

In some embodiments, prior to the collecting step, the pH is raised to a pH of 6. In some embodiments, the pH is raised to 6 using sodium hydroxide.

A illustrative method for producing a composition comprising a recombinant protein and a recombinant cell byproduct and separate the recombinant protein and the recombinant cell byproduct is shown in FIG. 6.

In embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In various embodiments, the composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic conditions. In some cases, protein-containing composition having a preferred pH and/or ionic conditions is further heat treated and/or dried. The heat treatment and/or drying step may produce a dry protein product having a reduced quantity of the off-flavor component.

In various embodiments, the protein product having a reduced quantity of the off-flavor component comprises an at least 50% reduction in off-flavor component quantity relative to the composition comprising a recombinant protein and a recombinant cell byproduct. In some cases, the protein product has an at least 75% reduction, at least 80% reduction, at least 90% reduction, or at least 95% reduction in the off-flavor component relative to the composition comprising a recombinant protein and a recombinant cell byproduct.

In embodiments, less than about 5%, less than about 1%, less than about 0.1%, or less than about 0.01% of the weight of the protein product comprises the off-flavor component. In some cases, the off-flavor component in the protein product is virtually undetectable to a standard consumer.

### Heat and Vacuum-Based Purification

An aspect of the present disclosure is a method for preparing a protein product having a reduced quantity of a recombinant cell byproduct. The method comprises steps of: obtaining a dry protein composition comprising a recombinant protein and a recombinant cell byproduct; processing the dry protein composition under conditions that separate the recombinant protein from the plurality of recombinant cell byproducts, wherein the processing step comprises maintaining the composition under heat and vacuum conditions; collecting the separated recombinant protein, thereby obtaining a protein product having a reduced quantity of the recombinant cell byproduct. In this method the recombinant cell byproduct is an off-flavor component. In various embodiments, the step of processing the composition comprises use of heat and vacuum, which separates the recombinant protein and the recombinant cell byproduct, wherein the heat is applied at a temperature and duration such that the recombinant cell byproduct is volatized and a gaseous recombinant cell byproduct is removable.

A illustrative method for producing a composition comprising a recombinant protein and a recombinant cell byproduct and separate the recombinant protein and the recombinant cell byproduct is shown in FIG. 6.

In embodiments, the composition may be agitated while the heat is applied.

In some embodiments, the vacuum may be applied contemporaneous with an application of heat. In some cases, the vacuum facilitates removal of the gaseous recombinant cell byproduct. In some instances, the vacuum is about 75 torr to about 150 torr.

In various cases, the composition is heated to about 50 to about 58° C. In some instances, the temperature is up to 80° C.

In embodiments, the dry protein composition comprising the recombinant protein and the recombinant cell byproduct was produced by fermentation of the recombinant cell.

In some embodiments, the dry protein composition comprising the recombinant protein and the recombinant cell byproduct was previously treated to remove spent biomass including recombinant cells.

In various embodiments, the dry protein composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic conditions. In some cases, protein-containing composition having a preferred pH and/or ionic conditions is further heat treated and/or dried. The heat treatment and/or drying step may produce a dry protein product having a reduced quantity of the off-flavor component.

In various embodiments, the protein product having a reduced quantity of the off-flavor component comprises an at least 50% reduction in off-flavor component quantity relative to the composition comprising a recombinant protein and a recombinant cell byproduct. In some cases, the protein product has an at least 75% reduction, at least 80% reduction, at least 90% reduction, or at least 95% reduction in the off-flavor component relative to the composition comprising a recombinant protein and a recombinant cell byproduct.

In embodiments, less than about 5%, less than about 1%, less than about 0.1%, or less than about 0.01% of the weight of the protein product comprises the off-flavor component. In some cases, the off-flavor component in the protein product is virtually undetectable to a standard consumer.

### Low Fouling Filtration-Based Purification

An aspect of the present disclosure is a method for preparing a recombinant protein concentrate for a consumable food product, the protein product having a reduced quantity of recombinant cell byproducts. The method comprises steps of: (1) obtaining a post-fermentation composition from a fermentation process used to produce recombinant egg protein, the post-fermentation composition comprising a cell culturing medium containing recombinant protein secreted by recombinant cells and recombinant cell byproducts; (2) processing the post-fermentation composition under conditions that separate the recombinant protein from the plurality of recombinant cell byproducts without using a resin for separation, thereby obtaining a separated recombinant protein; (3) concentrating the separated recombinant protein with a low fouling filtering system that filters the separated recombinant protein to produce a recombinant protein concentrate; and (4) collecting the recombinant protein concentrate comprising recombinant egg protein for an ingredient composition in a consumable food product.

Proteins derived from the fermentation and downstream processing of yeast and fungi transformed to express an illustrative protein (e.g., ovomucoid (OVD) or ovalbumin (OVA)) may contain off-flavor components and exopolysaccharide (EPS) due to the purification method. Powder proteins can be further purified to eliminate off-flavor and EPS compounds. The following example describes one such method. The concentration membranes typically used create a substantial challenge in terms of operation and product consistency due to fouling. To maintain a consistent product, low cost of cleaning, simpler operation, a low fouling membrane (Zwitterco Inc) may be used.

The low fouling membrane is synthesized with a zwitterionic surface coat that enables the water permeation, repels the protein from the surface and minimizes the concentration polarization layer formation. This takes the advantage of the zwitterionic surfaces enabling the flux to be constant throughout the process. Water-loving zwitterions are combined with a strong, hydrophobic backbone to get highly hydrophilic, net-neutral surface and pore structure that is highly resistant to fouling from organic molecules (e.g. fats, oils, proteins). This chemistry results in intrinsic pore uniformity, no ionic or other interactions at the surface, and high tolerance of salts, pH.

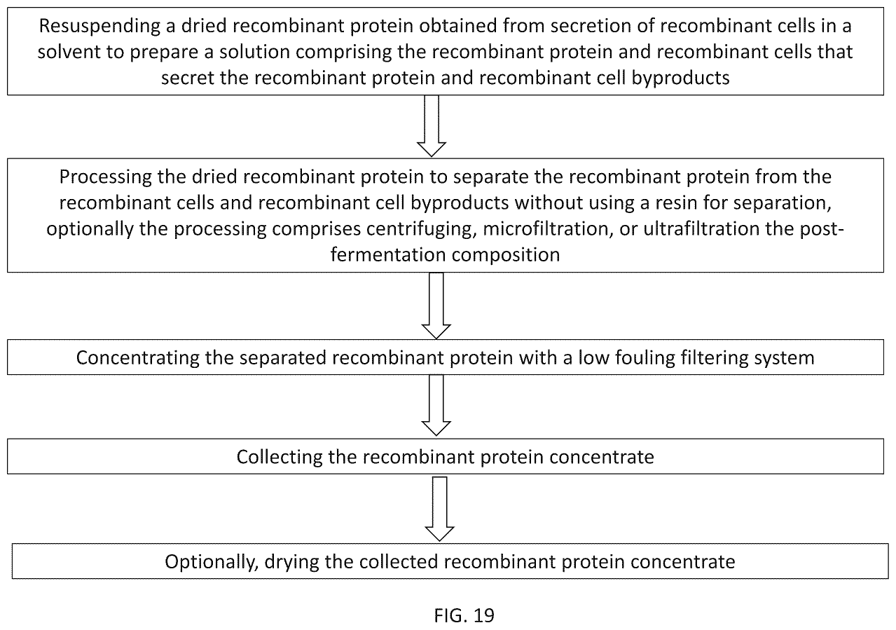

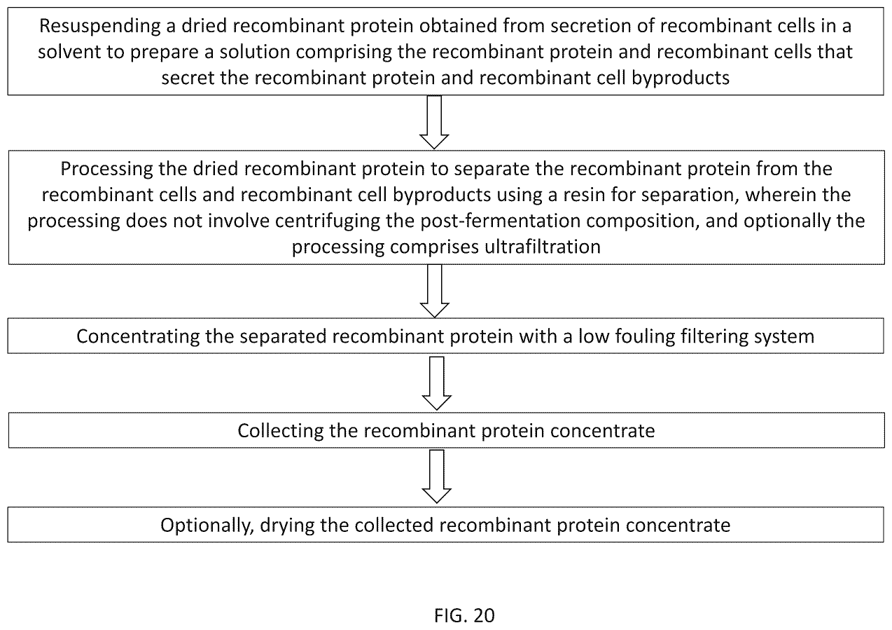

As a result, these membranes deliver consistent and higher flux than the conventional membrane and retain flux with mild maintenance washes without any irreversible fouling. Further, high consistent flux increases the concentration factor and enables case of implementing the continuous system. Also, a reduced or a milder cleaning directly results in the increase of the membrane life thereby reducing the operating costs substantially (chemicals, membranes) as well a greener process with lower wastewater burden on the system. Methods using low fouling filtration for the preparation of purified recombinant protein concentrate or dried recombinant protein are illustrated in FIGS. 19 and 20.

In some embodiments, the method reduces the quantity of exopolysaccharide(EPS) and/or the quantity of off-flavor components relative to the composition comprising a recombinant protein and the recombinant cell byproduct by at least 25% reduction, at least 30% reduction, an at least 35% reduction, at least 40% reduction, at least 45% reduction, at least 50% reduction, at least 55% reduction, at least 60% reduction, at least 65% reduction, at least 70% reduction, at least 75% reduction at least 75% reduction, at least 80% reduction, at least 90% reduction, or at least 95%.

In some embodiments, the method produces recombinant protein concentrate providing an improvement to at least one additional feature selected from the group consisting of flavor, moisture retention, water activity, mouthfeel, texture, hardness, cohesiveness, springiness, chewiness, stability to heat treatment, stability to pH, firmness, toughness, resilience, and stability for transportation.

### Protein Products and Protein-Containing Consumable Compositions

Another aspect of the present disclosure is a protein product prepared by any herein-disclosed method.

Yet another aspect of the present disclosure is a consumable composition comprising any-herein disclosed protein product.

In an aspect, the present disclosure provides a herein-disclosed consumable composition of for use in a food product.

In embodiments, the consumable composition further includes at least one consumable ingredient. In some cases, the consumable ingredient is a solvent, e.g., water, carbonated water, alcohol, juice, and any other commercially available drink.

In embodiments, the consumable composition comprising the protein product and having a reduced quantity of the recombinant cell byproduct has one or more different properties relative to an equivalent consumable composition that does not have a reduced quantity of the recombinant cell byproduct.

In embodiments, the properties include density, viscosity, gel hardness, chewiness, foam capacity, foam stability, solubility, clarity, texture, foaming, whipping, seeping, gelling, clarification, coagulation, coating, crystallization control, drying, edible packaging film, finishing, flavor, fortification, freezability, gloss, humectancy, insulation, moisturizing, mouthfeel, pH stability, protein enrichment, richness, shelf life extension, structure, tenderization, texture, thickening, water-binding, oil-binding, browning, emulsification, nitrogen:carbon ratio and/or anti-microbial activity. In some cases, the different property comprises a desirable increase in the property or the different property comprises a desirable decrease in the property.

An aspect of the present disclosure is a method for preparing a consumable composition. The method comprising steps of: obtaining a composition comprising a recombinant protein and a recombinant cell byproduct, wherein the recombinant cell byproduct is an exopolysaccharide (EPS) or an off-flavor component; processing the composition under conditions that separate the recombinant protein and the EPS or off-flavor component; collecting the separated recombinant protein, thereby obtaining a protein product having a reduced quantity of the EPS and/or collecting the separated EPS, thereby obtaining an EPS product having a reduced quantity of the recombinant protein; and formulating a consumable composition comprising the protein product or the EPS product. In this method the processing step comprises: i) a resin that reversibly attaches to the recombinant protein and does not substantially attach to the EPS, ii) an enzyme that digests the recombinant protein or the EPS, iii) an absorbent that reversibly attaches to the EPS and does not substantially attach to the recombinant protein, and/or iv) a flocculant that attaches to the EPS and does not substantially attach to the recombinant protein.

### Method for Collecting a Recombinant Cell Byproduct

In any of the herein-disclosed aspects or embodiments, a method may further comprise a step of collecting the separated recombinant cell byproduct. In some cases, the method further comprises a step of concentrating and/or purifying the separated recombinant cell byproduct, thereby obtaining an EPS product having a reduced quantity of the recombinant protein.

A illustrative method for producing a composition comprising a recombinant protein and a recombinant cell byproduct and separate the recombinant protein and the recombinant cell byproduct is shown in FIG. 5. The method of this aspect can be used to collect any extracellular product that is produced during a fermentation processes.

In an aspect, the present disclosure provides an exopolysaccharide (EPS) product produced by any herein-disclosed method.

In another aspect, the present disclosure provides a consumable composition comprising any herein-disclosed exopolysaccharide (EPS) product.

In embodiments, the consumable composition further includes at least one consumable ingredient.

In some embodiments, the consumable composition is for use as a food product.

In various embodiments, the EPS provides nutritional supplementation to a consumer.

In embodiments, the EPS improves gastrointestinal health to a consumer by preventing binding of pathogens to a consumer's digestive tract cell.

In some embodiments, the EPS improves gastrointestinal health to a consumer by promoting a favorable gut microbiome.

An aspect of the present disclosure is a method for preparing a consumable composition. The method comprising steps of: obtaining a composition comprising a recombinant protein and a recombinant cell byproduct, wherein the recombinant cell byproduct is an exopolysaccharide (EPS); processing the composition under conditions that separate the recombinant protein and the EPS; collecting the separated recombinant protein, thereby obtaining a protein product having a reduced quantity of the EPS and/or collecting the separated EPS, thereby obtaining an EPS product having a reduced quantity of the recombinant protein; and formulating a consumable composition comprising the protein product or the EPS product. In this method the processing step comprises: i) a resin that reversibly attaches to the recombinant protein and does not substantially attach to the EPS, ii) an enzyme that digests the recombinant protein or the EPS, iii) an absorbent that reversibly attaches to the EPS and does not substantially attach to the recombinant protein, and/or iv) a flocculant that attaches to the EPS and does not substantially attach to the recombinant protein.

### Features of Methods of the Present Disclosure

In embodiments, the ratio of recombinant cell byproduct to recombinant protein in the composition comprising a recombinant protein and a recombinant cell byproduct is about 1:3 to about 3:1. In some cases, the ratio is about 1:1.

In some embodiments, the protein product having a reduced quantity of the recombinant cell byproduct comprises an at least 50% reduction in recombinant cell byproduct quantity relative to the composition comprising a recombinant protein and a recombinant cell byproduct. In some cases, the protein product has an at least 75% reduction, at least 80% reduction, at least 90% reduction, or at least 95% reduction in recombinant cell byproduct quantity relative to the composition comprising a recombinant protein and a recombinant cell byproduct.

In various embodiments, less than about 10% of the weight of the protein product comprises the recombinant cell byproduct. In some cases, less than about 5% of the weight of the protein product comprises the recombinant cell byproduct.

In embodiments, the EPS or off-flavor component is generally inseparable from the recombinant protein when using size exclusion chromatography.

In some embodiments, the EPS or off-flavor component is naturally a component of a recombinant cell's cell wall. In some cases, the EPS or off-flavor component present in the composition comprising the recombinant protein and the recombinant cell byproduct was secreted from the recombinant cell rather than being incorporated into the recombinant cell's cell wall.

In various embodiments, the EPS has an apparent size of about 13 kDa to about 27 kDa as characterized by a size exclusion chromatography column.

In embodiments, the EPS comprises mannose. In some cases, the EPS further comprises N-acetylglucosamine and/or glucose.

In some embodiments, the EPS comprises about 91 mol % mannose, about 5 mol % N-acetylglucosamine, and about 3 mol % glucose as analyzed by gas chromatography in tandem with mass spectrometry. EPS can be quantified using a method using a pb binding column. An analytical HyperREZ XP Pb++ column (8 μm, 300×7.7 mm, Thermofisher Sci.) can be used for the measurement, which is eluted with water on UltiMate 3000 system (Thermofisher Sci.) operated at a flow rate of 0.6 mL/min and monitored with a refractive index detector.

In various embodiments, the EPS comprises an α(1,6)-linked backbone with α(1,2)-linked branches and/or α(1,3)-linked branches.

In embodiments, the EPS is a mannan.

In some embodiments, the recombinant cell is cell that expresses and/or secretes EPS and is selected from a fungal cell, such as filamentous fungus or a yeast, a bacterial cell, a plant cell, an insect cell, or a mammalian cell.

In various embodiments, the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor michci, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus. In some cases, the fungus is a Pichia species. In some cases, the Pichia species is Komagataella phaffii or Komagataella pastoris.

In embodiments, the recombinant protein is an enzyme, a nutritive protein, a food ingredient, or a food additive. In some cases, the enzyme is pepsinogen or pepsin.

In some embodiments, the protein is an egg-white protein. Eggs are almost an essential food component across the world. There is a huge market for eggs and egg ingredients. Eggs provide high protein and nutraceutical content and have been looked at as complete food in combination with milk. However, the eggs have a limited shelf life and are prone to bringing in infectious pathogens. People around the world specifically kids have been diagnosed with food allergies or have dietary restrictions inhibiting them to consume eggs. Also, to improve the productivity of the industrial scale production of eggs has a introduced use of growth hormones in addition to inhumane conditions for culturing chicken. The current egg substitutes have major limitations. None of the products extend the application to foaming as well as gelation. The product compositions in the package are unstable over time.

In some cases, the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof. In various cases, the egg-white protein is OVA, OVD, OVT, or OVL. In some cases, an egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

Any composition or method disclosed herein is applicable to any herein-disclosed composition or method. In other words, any aspect or embodiment described herein can be combined with any other aspect or embodiment as disclosed herein.

### Definitions

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Sequence identity, such as for the purpose of assessing percent complementarity, may be measured by any suitable alignment algorithm, including but not limited to the Needleman-Wunsch algorithm (see e.g., the EMBOSS Needle aligner available at the World Wide Web at ebi.ac.uk/Tools/psa/emboss_needle/nucleotide.html, optionally with default settings), the BLAST algorithm (see e.g., the BLAST alignment tool available at blast.ncbi.nlm.nih.gov/Blast.cgi, optionally with default settings), and the Smith-Waterman algorithm (see e.g., the EMBOSS Water aligner available at the World Wide Web at ebi.ac.uk/Tools/psa/emboss_water/nucleotide.html, optionally with default settings). Optimal alignment may be assessed using any suitable parameters of a chosen algorithm, including default parameters.

The term “bird” includes both domesticated birds and non-domesticated birds such as wildlife and the like. Birds include, but are not limited to, poultry, fowl, waterfowl, game bird, ratite (e.g., flightless bird), chicken (Gallus gallus domesticus), quail, turkey, duck, ostrich (Struthio camelus), Somali ostrich (Struthio molybdophanes), goose, gull, guineafowl, pheasant, emu (Dromaius novachollandiae), American rhea (Rhea americana), Darwin's rhea (Rhea pennata), and kiwi. Tissues, cells, and their progeny of a biological entity obtained in vivo or cultured in vitro are also encompassed. A bird may lay eggs.

As used herein, the terms “consumable composition” or “consumable product” refers to a composition or product, which comprises a recombinant protein or composition comprising recombinant protein and other ingredients and may be consumed (e.g., by eating, chewing, drinking, tasting, ingesting, or swallowing). Consumable products include food products, beverage products, dietary supplements, food additives, pharmaceutical products, and hygiene products, as non-limiting examples. Food products include, but are not limited to, baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings. Beverage products include, but are not limited to, soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks. Dietary supplements include multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement. A consumer of a consumable product or consumable composition is any animal, including domesticated animals (e.g., livestock) and humans.

Processing of a consumable product to form a processed consumable product may include, but is not limited to, freezing, chilling, heating, baking, roasting, broiling, boiling, blanching, packaging, canning, bleaching, enriching, drying, pressing, grinding, mixing, parcooking, cooking, proofing, marinating, cutting, slicing, dicing, crushing, shredding, chopping, shaking, coring, spiralizing, rolling, juicing, straining, filtering, kneading, whisking, beating, whipping, grating, stuffing, peeling, deseeding, smoking, curing, salting, preserving, pickling, fermenting, homogenizing, pasteurizing, sterilizing, stabilizing, blending, pureeing, fortifying, refining, hydrogenating, aging, extending shelf life, or adding enzymes.

As used herein, the term “solvent” refers to a liquid, which may be mixed with or used to dissolve a composition or one or more components of a composition such as a protein. Non-limiting examples of a solvent include water, ethanol, and isopropanol. The solvent can be potable. The solvent can be water. Non-limiting examples of water include purified water, distilled water, double distilled water, deionized water, distilled deionized water, drinking water, well water, tap water, spring water, bottled water, carbonated water, mineral water, flavored water, or any combination thereof. A solvent may be a combination of two or more distinct solvents.

### Additional Embodiments

Embodiment 1. A method for preparing a consumable composition, the method comprising steps of: obtaining a composition comprising a recombinant protein and a plurality of recombinant cell byproducts, wherein the recombinant cell byproducts comprise an exopolysaccharide (EPS) and an off-flavor component; processing the composition under conditions that separate the recombinant protein from the plurality of recombinant cell byproducts, wherein the processing step comprises an anionic resin that reversibly attaches to the recombinant protein and does not substantially attach to the plurality of recombinant cell byproducts; collecting the separated recombinant protein, thereby obtaining a protein product having a reduced quantity of the plurality of recombinant cell byproducts; and formulating a consumable composition comprising the protein product.

Embodiment 2. The method of Embodiment 1, wherein the composition comprising the recombinant protein and the plurality of recombinant cell byproducts is a cell culturing medium.

Embodiment 3. The method of Embodiment 1 or Embodiment 2, wherein the composition comprising the recombinant protein and the plurality of recombinant cell byproducts is a cell culture medium comprising recombinant cells that secreted the recombinant protein and the plurality of recombinant cell byproducts.

Embodiment 4. The method of Embodiment 1, wherein the composition comprising the recombinant protein and the plurality of recombinant cell byproducts lacks recombinant cells that secreted the recombinant protein and the plurality of recombinant cell byproducts.

Embodiment 5. The method of any one of Embodiments 1 to 4, wherein the composition comprising the recombinant protein and the plurality of recombinant cell byproducts has a pH greater than the isoelectric point (pI) of the recombinant protein.

Embodiment 6. The method of Embodiment 5, wherein the pH of the composition comprising the recombinant protein and the plurality of recombinant cell byproducts is not modified to achieve a pH greater than the pI of the recombinant protein.

Embodiment 7. The method of any one of Embodiments 1 to 6, wherein the anion resin is a strong anion exchange resin or a weak anion exchange resin.

Embodiment 8. The method of any one of Embodiments 1 to 7, wherein the anion resin is one or more of Capto Q resin, a DEAE type weak anion exchanger, a resin with trimethyl aminoethyl groups, a resin with triethyl aminoethyl groups, a resin with quaternary amine groups.

Embodiment 9. The method of any one of Embodiments 1 to 8, wherein the anion resin is a component of a chromatography system.

Embodiment 10. The method of Embodiment 9, wherein the chromatography system operates in batch mode with an axial flow column or a radial flow column or a centrifugal column or by use of a membrane chromatography column.

Embodiment 11. The method of Embodiment 9, wherein the chromatography system operates in a continuous mode comprising multiple columns in parallel, with the feed to the columns being switchable such that various steps in a chromatography process (e.g., equilibration, load, elute, and clean), occur contemporaneously.

Embodiment 12. The method of Embodiment 11, wherein the continuous mode comprises a simulated moving bed (SMB) or an Ion Separator (e.g., ISEP®) system.

Embodiment 13. The method of any one of Embodiments 4 to 12, wherein the composition comprising the recombinant protein and the plurality of recombinant cell byproducts was previously treated to remove spent biomass including recombinant cells and/or was previously treated to remove small non-protein molecules.

Embodiment 14. The method of Embodiment 13, wherein the treatment to remove small non-protein molecules comprises a step that concentrates the composition comprising the recombinant protein and the plurality of recombinant cell byproducts.

Embodiment 15. The method of Embodiment 13 or Embodiment 14, wherein the treatment to remove small non-protein molecules comprises a diafiltration buffer.

Embodiment 16. The method of any one of Embodiments 1 to 15 further comprising a concentration step and/or diafiltration treatment of the separated recombinant protein to produce a protein-containing composition having a preferred pH and/or ionic condition.

Embodiment 17. The method of Embodiment 16, wherein the protein-containing composition having a preferred pH and/or ionic condition is further heat treated and/or dried.

Embodiment 18. The method of any one of Embodiments 1 to 15, wherein the composition comprising the recombinant protein and the plurality of recombinant cell byproducts and/or the protein product having a reduced quantity of the plurality of recombinant cell byproducts is further heat treated and/or dried.

Embodiment 19. The method of Embodiment 17 or Embodiment 18, wherein the heat treatment separates the recombinant protein and the off-flavor component, wherein the heat is applied at a temperature and duration such that the off-flavor component is volatized and a gaseous off-flavor component is removable.

Embodiment 20. The method of Embodiment 19, wherein a vacuum is applied contemporaneous with the application of heat and the vacuum facilitates removal of the gaseous off-flavor component.

Embodiment 21. The method of any one of Embodiments 1 to 20, wherein the off-flavor component is an acid, an alcohol, an aldehyde, an aromatic, an ester, or a ketone.

Embodiment 22. The method of any one of Embodiments 1 to 21, wherein the off-flavor component is (E)-2-nonenal; 1-dodecene; 1-hexanol, 2-ethyl-; 1-hexen-3-one; 1-octen-3-one; 2,3-butanedione; 2-butanone; 2-methylbutanal; 2-methylpropanal; 2-propanone; 2-undecanone; 3-methylbutanal; acetaldehyde; benzene ethanol; benzyl alcohol; butanal, 3-methyl-; chlorotoluene; nonanoic acid; p-cresol; or propanoic acid, 2-methyl-, 3-hydroxy-2,4,4-trimethylpentyl ester.

Embodiment 23. The method of any one of Embodiments 17 to 22, wherein the temperature of the protein-containing composition having a preferred pH and/or ionic conditions, the composition comprising the recombinant protein and the plurality of recombinant cell byproducts, and/or the protein product having a reduced quantity of the plurality of recombinant cell byproducts during the heat treatment is up to 80° C., e.g., from about 50° C. to about 60° C.

Embodiment 24. The method any one of Embodiments 17 to 23, wherein the method comprises agitation during the heat treatment.

Embodiment 25. The method any one of Embodiments 17 to 24, wherein the heat treatment and/or drying step produces a dry protein product having a reduced quantity of the plurality of recombinant cell byproducts.

Embodiment 26. The method of any one of Embodiments 1 to 25, wherein the composition comprising the recombinant protein and the plurality of recombinant cell byproducts and/or the protein product having a reduced quantity of the plurality of recombinant cell byproducts further undergoes an oxidation step, e.g., comprising the addition of hydrogen peroxide.

Embodiment 27. The method of any one of Embodiments 1 to 26, wherein the ratio of the recombinant cell byproducts to recombinant protein in the composition comprising a recombinant protein and the plurality of recombinant cell byproducts is about 1:3 to about 3:1.

Embodiment 28. The method of Embodiment 27, wherein the protein product has an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction an at least 75% reduction, at least 80% reduction, at least 90% reduction, or at least 95% reduction in the quantity of EPS and/or the quantity of off-flavor components relative to the composition comprising a recombinant protein and the plurality of recombinant cell byproducts.

Embodiment 29. The method of Embodiment 28, wherein less than about 10% of the weight of the protein product comprises recombinant cell byproducts.

Embodiment 30. The method of any one of Embodiments 1 to 29, wherein less than about 5% of the weight of the protein product comprises recombinant cell byproducts.

Embodiment 31. The method of any one of Embodiments 1 to 30, wherein less than about 5%, less than about 1%, less than about 0.1%, or less than about 0.01% of the weight of the protein product comprises the off-flavor component.

Embodiment 32. The method of any one of Embodiments 1 to 31, wherein the off-flavor component in the protein product is virtually undetectable to a standard consumer.

Embodiment 33. The method of any one of Embodiments 1 to 32, wherein the EPS is generally inseparable from the recombinant protein when using size exclusion chromatography.

Embodiment 34. The method of any one of Embodiments 1 to 33, wherein the EPS is naturally a component of a recombinant cell's cell wall.

Embodiment 35. The method of one of Embodiments 1 to 34, wherein the EPS has an apparent size of about 13 kDa to about 27 kDa as characterized by a size exclusion chromatography column.

Embodiment 36. The method of any one of Embodiments 1 to 35, wherein the EPS comprises mannose.

Embodiment 37. The method of any one of Embodiments 1 to 36, wherein the EPS further comprises N-acetylglucosamine and/or glucose.

Embodiment 38. The method of any one of Embodiments 1 to 37, wherein the EPS comprises about 91 mol % mannose, about 5 mol % N-acetylglucosamine, and about 3 mol % glucose as analyzed by gas chromatography in tandem with mass spectrometry.

Embodiment 39. The method of any one of Embodiments 1 to 38, wherein the EPS comprises an α(1,6)-linked backbone with α(1,2)-linked branches and/or α(1,3)-linked branches.

Embodiment 40. The method of any one of Embodiments 1 to 39, wherein the EPS is a mannan.

Embodiment 41. The method of any one of Embodiments 1 to 40, wherein the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

Embodiment 42. The method of any one of Embodiments 1 to 41, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

Embodiment 43. The method of Embodiment 41 or Embodiment 42, wherein the fungus is a Pichia species.

Embodiment 44. The method of Embodiment 43, wherein the Pichia species is Komagataella phaffii or Komagataella pastoris.

Embodiment 45. The method of any one of Embodiments 1 to 44, wherein the recombinant protein is an enzyme, a nutritive protein, a food ingredient, or a food additive.

Embodiment 46. The method of Embodiment 45, wherein the enzyme is pepsinogen or pepsin.

Embodiment 47. The method of Embodiment 45, wherein the protein is an egg-white protein.

Embodiment 48. The method of Embodiment 47, wherein the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

Embodiment 49. The method of Embodiment 47 or Embodiment 48, wherein the egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

Embodiment 50. The method of any one of Embodiments 1 to 49, wherein the consumable composition comprising the protein product comprises food products, beverage products, or dietary supplements.

Embodiment 51. The method of Embodiment 50, wherein the food products comprise baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings.

Embodiment 52. The method of Embodiment 50, wherein the beverage products comprise soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks.

Embodiment 53. The method of Embodiment 50, wherein the dietary supplements comprise multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement.

Embodiment 54. The method of any one of Embodiments 1 to 53, wherein the processing the composition under conditions that separate the recombinant protein from the plurality of recombinant cell byproducts further comprises one or more of: i) one or more cation exchange resin that reversibly attach to the recombinant protein and does not substantially attach to the EPS, ii) an enzyme that digests the recombinant protein or the EPS, iii) an adsorbent that reversibly attaches to the EPS and does not substantially attach to the recombinant protein, and/or iv) a flocculant that attaches to the EPS and does not substantially attach to the recombinant protein.

Embodiment 55. A consumable composition obtained by the method of any one of Embodiments 1 to 54.

Embodiment 56. A method for preparing a consumable composition, the method comprising steps of: obtaining a composition comprising a recombinant protein and a plurality of recombinant cell byproducts, wherein the recombinant cell byproducts comprise an exopolysaccharide (EPS) and an off-flavor component; processing the composition under conditions that separate the recombinant protein from the plurality of recombinant cell byproducts, wherein the processing step comprises one or more cation exchange resins that reversibly attach to the recombinant protein and do not substantially attach to the plurality of recombinant cell byproducts collecting the separated recombinant protein, thereby obtaining a protein product having a reduced quantity of the plurality of recombinant cell byproducts; and formulating a consumable composition comprising the protein product.

Embodiment 58. The method of Embodiment 56 or Embodiment 57, wherein the one or more cation exchange resins comprise poly styrene divinyl benzene, poly methacrylate or cellulose or cross-linked dextran or cross-linked agarose or inorganic materials coated with hydrophilic polymers.

Embodiment 59. The method of any one of Embodiments 56 to 58, wherein the one or more cation exchange resins have a particle size of from about 50 μm and about 200 μm and/or have a protein binding capacity of from about 50 to about 100 g protein/L resin.

Embodiment 60. The method of any one of Embodiments 56 to 59, wherein the one or more cation exchange resins comprise Cytiva Capto S, HP20, resindion SP400, Sepragen S, SP20, and/or Mitsubishi Relisorb EXE349.

Embodiment 61. The method of any one of Embodiments 56 to 60, wherein the processing step comprises two cationic resins, wherein the two cationic resins are in a ratio of 1:5 to 5:1, 1:4 to 4:1, 1:3 to 3:1, 1:2 to 2:1, or 1:1.

Embodiment 62. The method of Embodiment 61, wherein the two resins are SP400 and Sepragen S and in a ratio of about 3:1, e.g., 2.75:1.25.

Embodiment 63. The method of any one of Embodiments 56 to 62, wherein the composition comprising the recombinant protein and the plurality of recombinant cell byproducts lacks recombinant cells that secreted the recombinant protein and the plurality of recombinant cell byproducts.

Embodiment 64. The method of any one of Embodiments 56 to 63, wherein the composition comprising the recombinant protein and the plurality of recombinant cell byproducts has a pH less than the isoelectric point (pI) of the recombinant protein, which is achieved by lowering the pH of the composition comprising the recombinant protein and the plurality of recombinant cell byproducts.

Embodiment 65. The method of any one of Embodiments 56 to 64, wherein the one or more cationic resins are components of a chromatography system, wherein the chromatography system operates in batch mode with an axial flow column or a radial flow column or a centrifugal column or by use of a membrane chromatography column.

Embodiment 66. The method of any one of Embodiments 56 to 64, wherein the one or more cationic resins are components of a chromatography system, wherein the chromatography system operates in a continuous mode comprising multiple columns in parallel, with the feed to the columns being switchable such that various steps in a chromatography process (e.g., equilibration, load, elute, and clean), occur contemporaneously.

Embodiment 67. The method of Embodiment 66, wherein the continuous mode comprises a simulated moving bed (SMB) or an Ion Separator (e.g., ISEP®) system.

Embodiment 68. The method of any one of Embodiments 62 to 67, wherein the composition comprising the recombinant protein and the plurality of recombinant cell byproducts was previously treated to remove spent biomass including recombinant cells and/or was previously treated to remove small non-protein molecules.

Embodiment 69. The method of Embodiment 68, wherein the treatment to remove small non-protein molecules comprises a step that concentrates the composition comprising the recombinant protein and the plurality of recombinant cell byproducts.

Embodiment 70. The method of Embodiment 68 or Embodiment 69, wherein the treatment to remove small non-protein molecules comprises a diafiltration buffer.

Embodiment 71. The method of any one of Embodiments 56 to 70 further comprising a concentration step and/or diafiltration treatment of the separated recombinant protein to produce a protein-containing composition having a preferred pH and/or ionic condition.

Embodiment 72. The method of Embodiment 71, wherein the protein-containing composition having a preferred pH and/or ionic condition is further heat treated and/or dried.

Embodiment 73. The method of any one of Embodiments 56 to 70, wherein the composition comprising the recombinant protein and the plurality of recombinant cell byproducts and/or the protein product having a reduced quantity of the plurality of recombinant cell byproducts is further heat treated and/or dried.

Embodiment 74. The method of Embodiment 72 or Embodiment 73, wherein the heat treatment separates the recombinant protein and the off-flavor component, wherein the heat is applied at a temperature and duration such that the off-flavor component is volatized and a gaseous off-flavor component is removable.

Embodiment 75. The method of Embodiment 74, wherein a vacuum is applied contemporaneous with the application of heat and the vacuum facilitates removal of the gaseous off-flavor component.

Embodiment 76. The method of any one of Embodiments 56 to 75, wherein the off-flavor component is an acid, an alcohol, an aldehyde, an aromatic, an ester, or a ketone.

Embodiment 77. The method of any one of Embodiments 56 to 76, wherein the off-flavor component is (E)-2-nonenal; 1-dodecene; 1-hexanol, 2-ethyl-; 1-hexen-3-one; 1-octen-3-one; 2,3-butanedione; 2-butanone; 2-methylbutanal; 2-methylpropanal; 2-propanone; 2-undecanone; 3-methylbutanal; acetaldehyde; benzene ethanol; benzyl alcohol; butanal, 3-methyl-; chlorotoluene; nonanoic acid; p-cresol; or propanoic acid, 2-methyl-, 3-hydroxy-2,4,4-trimethylpentyl ester.

Embodiment 78. The method of any one of Embodiments 72 to 77, wherein the temperature of the protein-containing composition having a preferred pH and/or ionic conditions, the composition comprising the recombinant protein and the plurality of recombinant cell byproducts, and/or the protein product having a reduced quantity of the plurality of recombinant cell byproducts during the heat treatment is up to 80° C., e.g., from about 50° C. to about 60° C.

Embodiment 79. The method any one of Embodiments 72 to 78, wherein the method comprises agitation during the heat treatment.

Embodiment 80. The method any one of Embodiments 72 to 79, wherein the heat treatment and/or drying step produces a dry protein product having a reduced quantity of the plurality of recombinant cell byproducts.

Embodiment 81. The method of any one of Embodiments 56 to 80, wherein the composition comprising the recombinant protein and the plurality of recombinant cell byproducts and/or the protein product having a reduced quantity of the plurality of recombinant cell byproducts further undergoes an oxidation step, e.g., comprising the addition of hydrogen peroxide.

Embodiment 82. The method of any one of Embodiments 56 to 81, wherein the ratio of the recombinant cell byproducts to recombinant protein in the composition comprising a recombinant protein and the plurality of recombinant cell byproducts is about 1:3 to about 3:1.

Embodiment 83. The method of Embodiment 82, wherein the protein product has an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction an at least 75% reduction, at least 80% reduction, at least 90% reduction, or at least 95% reduction in the quantity of EPS and/or the quantity of off-flavor components relative to the composition comprising a recombinant protein and the plurality of recombinant cell byproducts.

Embodiment 84. The method of Embodiment 83, wherein less than about 10% of the weight of the protein product comprises recombinant cell byproducts.

Embodiment 85. The method of any one of Embodiments 56 to 84, wherein less than about 5% of the weight of the protein product comprises recombinant cell byproducts.

Embodiment 86. The method of any one of Embodiments 56 to 85, wherein less than about 5%, less than about 1%, less than about 0.1%, or less than about 0.01% of the weight of the protein product comprises the off-flavor component.

Embodiment 87. The method of any one of Embodiments 56 to 86, wherein the off-flavor component in the protein product is virtually undetectable to a standard consumer.

Embodiment 88. The method of any one of Embodiments 56 to 87, wherein the EPS is generally inseparable from the recombinant protein when using size exclusion chromatography.

Embodiment 89. The method of any one of Embodiments 56 to 88, wherein the EPS is naturally a component of a recombinant cell's cell wall.

Embodiment 90. The method of one of Embodiments 56 to 89, wherein the EPS has an apparent size of about 13 kDa to about 27 kDa as characterized by a size exclusion chromatography column.

Embodiment 91. The method of any one of Embodiments 56 to 90, wherein the EPS comprises mannose.

Embodiment 92. The method of any one of Embodiments 56 to 91, wherein the EPS further comprises N-acetylglucosamine and/or glucose.

Embodiment 93. The method of any one of Embodiments 56 to 92, wherein the EPS comprises about 91 mol % mannose, about 5 mol % N-acetylglucosamine, and about 3 mol % glucose as analyzed by gas chromatography in tandem with mass spectrometry.

Embodiment 94. The method of any one of Embodiments 56 to 93, wherein the EPS comprises an α(1,6)-linked backbone with α(1,2)-linked branches and/or α(1,3)-linked branches.

Embodiment 95. The method of any one of Embodiments 56 to 94, wherein the EPS is a mannan.

Embodiment 96. The method of any one of Embodiments 56 to 95, wherein the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

Embodiment 97. The method of any one of Embodiments 56 to 96, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor michei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

Embodiment 98. The method of Embodiment 96 or Embodiment 97, wherein the fungus is a Pichia species.

Embodiment 99. The method of Embodiment 98, wherein the Pichia species is Komagataella phaffii or Komagataella pastoris.

Embodiment 100. The method of any one of Embodiments 56 to 99, wherein the recombinant protein is an enzyme, a nutritive protein, a food ingredient, or a food additive.

Embodiment 101. The method of Embodiment 100, wherein the enzyme is pepsinogen or pepsin.

Embodiment 102. The method of Embodiment 100, wherein the protein is an egg-white protein.

Embodiment 103. The method of Embodiment 102, wherein the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

Embodiment 104. The method of Embodiment 102 or Embodiment 103, wherein the egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

Embodiment 105. The method of any one of Embodiments 56 to 104, wherein the consumable composition comprising the protein product comprises food products, beverage products, or dietary supplements.

Embodiment 106. The method of Embodiment 105, wherein the food products comprise baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings.

Embodiment 107. The method of Embodiment 105, wherein the beverage products comprise soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks.

Embodiment 108. The method of Embodiment 105, wherein the dietary supplements comprise multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement.

Embodiment 109. The method of any one of Embodiments 56 to 108, wherein the processing the composition under conditions that separate the recombinant protein from the plurality of recombinant cell byproducts further comprises one or more of: i) an anionic resin that reversibly attaches to the recombinant protein and does not substantially attach to the EPS, ii) an enzyme that digests the recombinant protein or the EPS, iii) an adsorbent that reversibly attaches to the EPS and does not substantially attach to the recombinant protein, and/or iv) a flocculant that attaches to the EPS and does not substantially attach to the recombinant protein.

Embodiment 110. A consumable composition obtained by the method of any one of Embodiments 56 to 109.

Embodiment 111. A method for preparing a consumable composition, the method comprising steps of:
obtaining a composition comprising a recombinant protein and a plurality of recombinant cell byproducts, wherein the recombinant cell byproducts comprise an exopolysaccharide (EPS) and an off-flavor component;
processing the composition under conditions that separate the recombinant protein from the plurality of recombinant cell byproducts, wherein the processing step comprises an flocculant that reversibly attaches to one or more components of the plurality of recombinant cell byproducts and does not substantially attach to the recombinant protein;
collecting the separated recombinant protein, thereby obtaining a protein product having a reduced quantity of the plurality of recombinant cell byproducts; and
formulating a consumable composition comprising the protein product.

Embodiment 112. A method for preparing a consumable composition, the method comprising steps of:
obtaining a composition comprising a recombinant protein and a plurality of recombinant cell byproducts, wherein the recombinant cell byproducts comprise an exopolysaccharide (EPS) and an off-flavor component;
processing the composition under conditions that separate the recombinant protein from the plurality of recombinant cell byproducts, wherein the processing step comprises an adsorbent that reversibly attaches to one or more components of the plurality of recombinant cell byproducts and does not substantially attach to the recombinant protein;
collecting the separated recombinant protein, thereby obtaining a protein product having a reduced quantity of the plurality of recombinant cell byproducts; and
formulating a consumable composition comprising the protein product.

Embodiment 113. A method for preparing a consumable composition, the method comprising steps of: obtaining a composition comprising a recombinant protein and a plurality of recombinant cell byproducts, wherein the recombinant cell byproducts comprise an exopolysaccharide (EPS) and an off-flavor component; processing the composition under conditions that separate the recombinant protein from the plurality of recombinant cell byproducts, wherein the processing step comprises an enzyme that either digests the recombinant protein or digests the EPS; collecting the separated recombinant protein, thereby obtaining a protein product having a reduced quantity of the plurality of recombinant cell byproducts; and formulating a consumable composition comprising the protein product.

Additionally, of the above embodiments described herein can be combined with any other embodiment as disclosed above.

### EXAMPLES

### Example 1: Use of a Resin for Preparing a Protein Product Having a Reduced Quantity of a Recombinant Cell Byproduct

A method of the present disclosure (e.g., as shown in FIG. 1) was used to prepare a protein product having a reduced quantity of a recombinant cell byproduct. In this example, a recombinant egg-white protein was used.

Table 1 (below) shows a typical composition comprising a recombinant protein and a recombinant cell byproduct before and after the use of resin-based (e.g., chromatography) purification process to reduce the quantity of the recombinant cell byproduct.

TABLE 1
Comparison of physio chemical properties
of the concentrated protein before
and after the purification step.

| | Concentrated | Purified |
|---|---|---|
| | protein | protein |
| Protein | 53 | 98 |
| (% w/w) | | |
| EPS | 47 | 2 |
| (% w/w) | | |

The purified protein was further used to demonstrate unique gelling properties similar to commercial egg whites thereby enabling formulation replacing eggs in the egg-based recipes.

Table 2, below, shows a comparison of the concentrated composition (comprising the recombinant protein and a recombinant cell byproduct) and purified protein product (which has reduced quantity of the recombinant cell byproduct) further for various functional properties is shown in the following table.

TABLE 2
Comparison of functional properties
of the concentrated protein before
and after the purification step.

| | Concentrated | Purified |
|---|---|---|
| | protein | protein |
| Gel hardness | High | low |
| Chewiness | High | Low |
| Foam capacity | High | High |
| Foam stability | Low | high |

Notably, removing the recombinant cell byproduct (e.g., EPS) reduced the gel hardness and chewiness of an illustrative consumable composition/food product relative.

Table 3, below, shows a comparison of the functional properties of the concentrated composition (comprising the recombinant protein and a recombinant cell byproduct) and a commercial-available egg substitute further for various functional properties.

TABLE 3
Comparison of functional properties of the
concentrated composition (comprising the
recombinant protein and a recombinant cell
byproduct) to a commercial egg white protein

| | Concentrated | Egg white |
|---|---|---|
| | protein | protein |
| Gel hardness | High | High |
| Chewiness | High | High |
| Foam capacity | High | High |

Surprisingly, it was discovered that the gelation characteristics of the concentrated composition (comprising the recombinant protein and a recombinant cell byproduct) was equivalent to an egg white powder, indicating significant contribution of the impurities towards gelation. Further, the foaming and foam retention properties as shown in Table 2. Thus, for high foam applications, it may be preferable to specifically modulate the quantities of the recombinant cell byproducts in a consumable composition.

A purified recombinant ovalbumin protein product was combined with various amounts of the recombinant cell byproduct (e.g., EPS or off-flavor component) to determine changes in product properties.

Resins with sulfopropyl, sulfomethyl, sulfonate may be used in this method. The backbone is typically a nonprotein binding material such as methacrylate or cellulose with typical particle size between 50-200 μm. The ligand density would accommodate protein binding capacity between 50-100 g protein/L resin.

### Example 2: Another Use of a Resin for Preparing a Protein Product Having a Reduced Quantity of a Recombinant Cell Byproduct

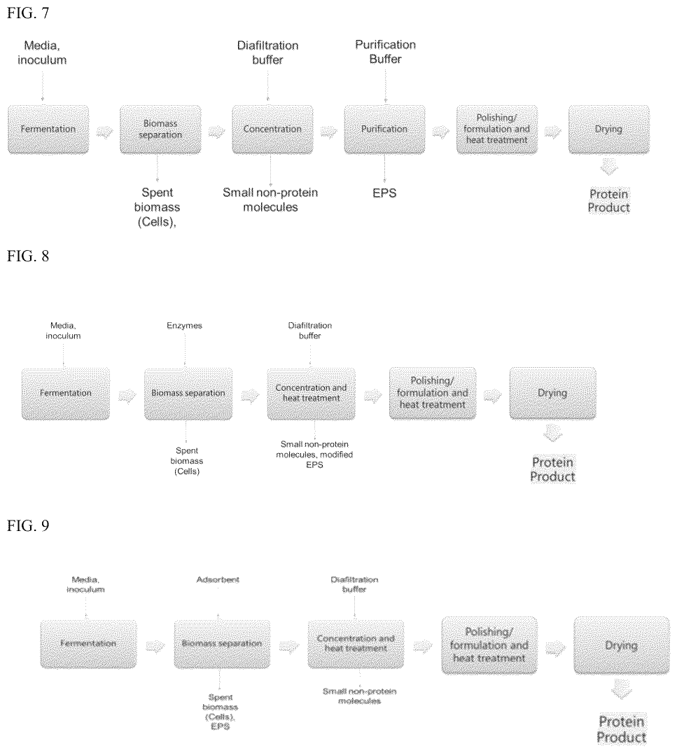

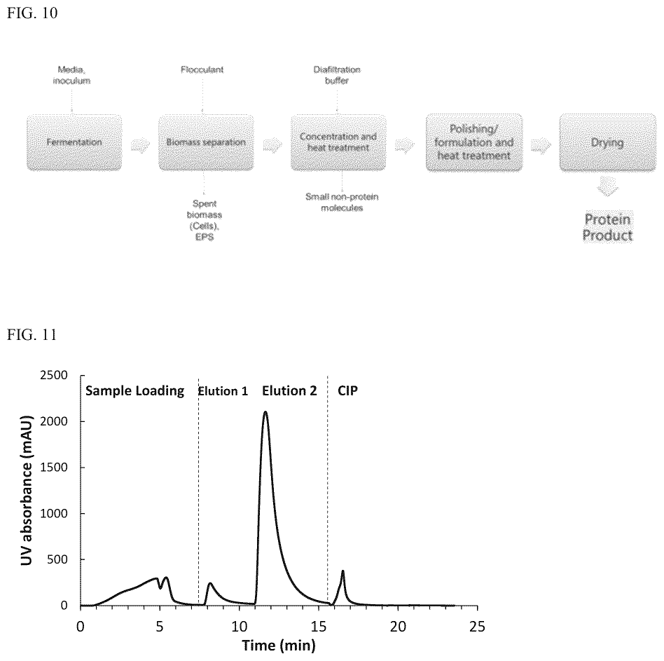

Another method of the present disclosure (e.g., as shown in FIG. 1 or FIG. 7) was used to prepare a protein product having a reduced quantity of a recombinant cell byproduct. In this example, a recombinant egg-white protein was used.

Pichia Pastoris strain derived from the historic Phillips Petroleum strain NRRL Y-11430 was designed to generate a nonmethanol-utilization (mutM) phenotype with transformations to express an illustrative protein, here ovomucoid, and a strong methanol inducible promoter. These transformant strains were further modified by adding a surface display enzyme that would reduce the complex carbohydrate to filterable size. Sequencing confirmed that this strain did not contain any antibiotic markers or prokaryotic vector origin of replication sequences.

The resulting strain was grown in fermentation conditions in high-density growth conditions at about pH 5. After about 36 hours of growth under fermentation conditions, the pH was raised to about pH 6, and expression of the illustrative protein was induced by the addition of methanol to the culture. The fermentation broth was centrifuged (using a bench centrifuge—Avant J18 rotor Bechman Coulter) to remove cells. This was followed by filtration of the supernatant using a 0.2 μm hollow fiber membrane filtration to remove host protein and cell debris. The protein solution was then concentrated using a 5 kDa ultrafiltration membrane to over 30 g/L protein and diafiltered extensively to remove most of the organic and inorganic impurities. The resulting protein concentrate was adjusted to pH 3.5 using citrate and loaded on to a chromatography column. This column was packed with Cation exchange resin (SP400, Mitsubishi Chemicals, Japan). The chromatography steps were carried out with an AKTA Explorer 900 (GE Healthcare Life Sciences) and the Unicorn interface software (version 5.11) at approximately 22° C. in a down-flow mode, the chromatography method generally consisted of an equilibration step, a load (flowthrough) step, a wash step to remove non-bound protein, an elution step to remove product, a cleaning in place (CIP) step and regeneration step. The column volumes of elution, at each step and the buffer used is shown in the following Table 4.

TABLE 4
List of buffers and the column volumes
required in a typical process.

| | Number of | |
|---|---|---|
| | Column | Buffer |
| Step | Volumes | composition |
| Equilibration | 4 | 25 mM Citrate buffer |
| Sample | 1-2 | Feed sample |
| application | | |
| Column Wash | 2-3 | 25 mM Citrate buffer |
| Elution | 3-4 | 25 mM Citrate buffer |
| | | with 1 M NaCl |
| CIP | 3 | 1 M NaOH |
| Regeneration | 3 | 20% EtOH and 150 |
| | | mM NaCl |

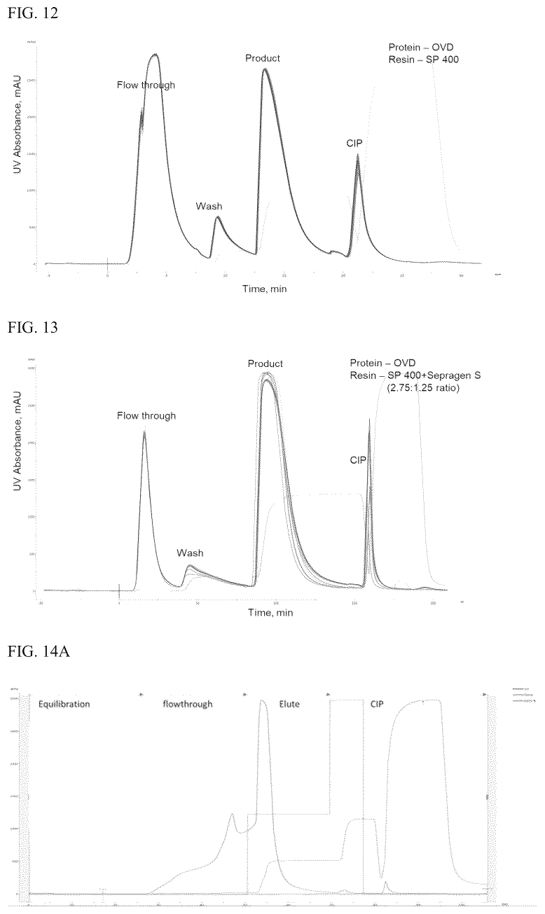

The elution profile is shown in FIG. 12.

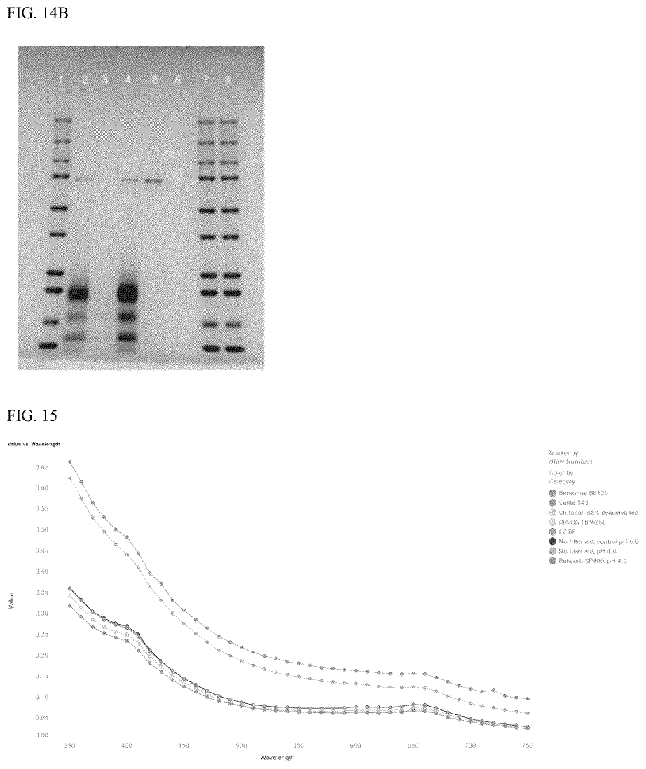

In this and other examples, the following terms are used: “Equilibration” is preloading a column, “Flow through” is the material passing the column when the UF diafiltered concentrate is passed through it and shown as lane 3 on the gel and minimal product loss, “Elute” is the protein of interest rich fraction (e.g., in the gel of FIG. 14, in lane 4) and the biggest peak in the chromatogram, “CIP” is the cleaning solution elution.

In an alternate method, the column is packed with a unique mixture of two resins. SP400 (Mitsubishi Chemicals, Japan) and Sepragen S (Sepragen, California) in the ratio of 2.75:1.25. The buffer compositions and the column volumes are maintained as shown in Table 4. The elution profile is shown in FIG. 13.

Resins with sulfopropyl, sulfomethyl, sulfonate may be used in this method. The backbone is typically a nonprotein binding material such as methacrylate or cellulose with typical particle size between 50-200 μm. The ligand density would accommodate protein binding capacity between 50-100 g protein/L resin.

### Example 3: An Anionic Resin for Preparing a Protein Product Having a Reduced Quantity of a Recombinant Cell Byproduct

In Example 2, the column was packed with a cationic resin. In the present example, the column is packed with anionic exchange capto Q resin (Cytiva Chemicals). The buffer compositions and the column volumes are maintained as shown in Table 5. Note that the feed is not pH modified here simplifying the process significantly. Note that the feed is not pH modified here simplifying the process significantly.

TABLE 5
List of buffers and the column volumes required
in a typical process

| | Number of | |
|---|---|---|
| | Column | |
| Step | Volumes | Buffer composition |
| Equilibration | 4 | 25 mM Sodium Phosphate + |
| | | 16 mM Sodium Chloride (pH 6; |
| | | Conductivity-3.4 mS/cm) |
| Sample | 1-2 | Feed sample diluted to |
| application | | 3.4 mS/cm |
| Column Wash | 2-3 | Same as equilibration |
| Elution | 3-4 | 25 mM Sodium Phosphate + |
| | | 300 mM Sodium Chloride |
| CIP | 3 | 1 M NaOH |
| Regeneration | 3 | 20% EtOH and 16 mM NaCl |

The elution profile is shown in FIG. 14A and a gel showing the protein fractions is shown in FIG. 14B.

Resins with trimethyl aminoethyl, triethyl aminoethyl, quaternary amine groups may be used in methods described in this example.

### Example 4: Use of an Adsorbent or Flocculant for Preparing a Protein Product Having a Reduced Quantity of a Recombinant Cell Byproduct

The protein concentrate prepared as per Example 3 above was further tested with multiple adsorbents and flocculants. The procedure used was as follows: 0.05 g of the test adsorbent or flocculant was weighed out in a 50 mL falcon tube. 5 mL of protein concentrate was added to the test adsorbent or flocculant. The tubes were then placed on roller shaker for 1 hour followed by centrifuging at 3214 g for 30 min. The supernatant was then tested for absorbance, protein and EPS. Each material was tested in duplicate. The list of adsorbents and flocculants tested is as follows. Control (pH 6), control (pH 4), Celite 545, Bentonite BE125, DIAION HPA25L, Chitosan 85% deacetylated, EZ DE (diatomaceous earth), ultrapure diatomaceous earth, Relisorb SP400 (pH 4). The absorbance spectra for this study is shown in FIG. 15. Notably, Relisorb did not work since it bound the protein rather than the impurity at pH 4.0. At higher pH this resin is expected to elute everything.

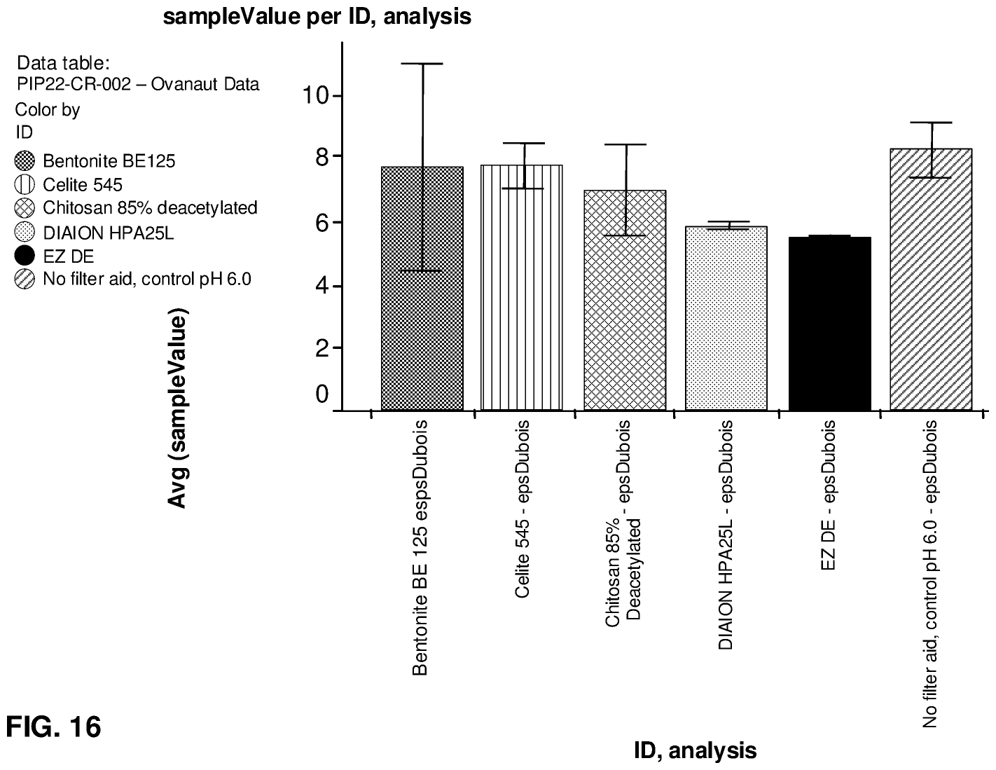

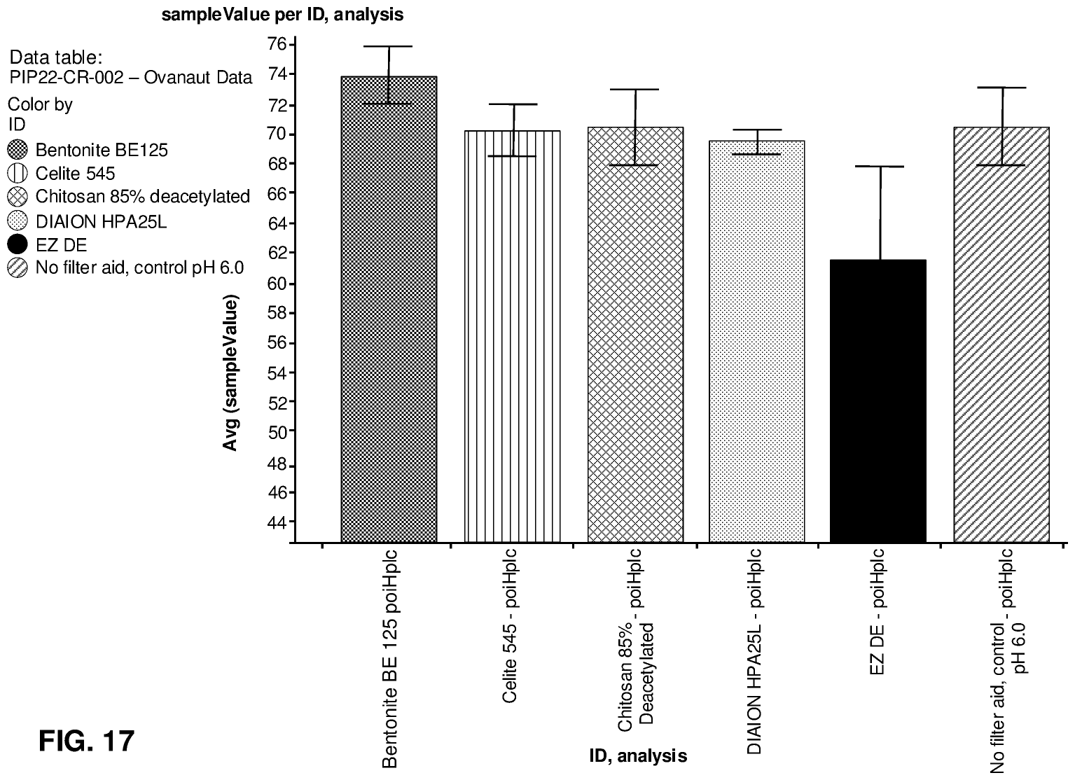

Further data is shown in FIG. 16 and FIG. 17. These figures show that compared to the control (black) the HPA25L and chitosan adsorb more EPS and almost no protein whereas at pH 4 the SP 400 adsorbs protein and not EPS. Diatomaceous earth (DE) adsorbs both protein and EPS with no differentiation.

### Example 5: Use of Flocculants to Extract EPS

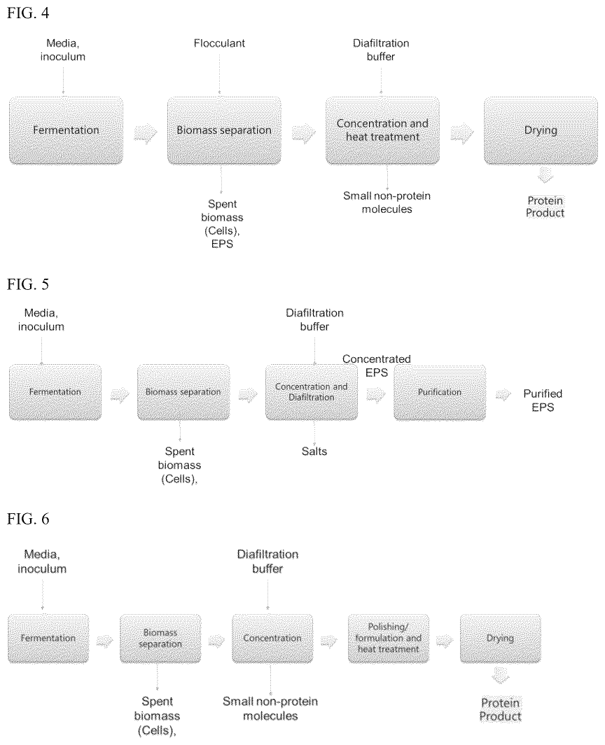

A Pichia Pastoris strain is grown in fermentation conditions in high-density growth conditions at about pH 5. After about 36 hours of growth under fermentation conditions, the pH is raised to about pH 6, and expression of a protein of interest is induced by the addition of methanol to the culture. At the end of the fermentation, a flocculant is added to the broth at a concentration of 10-100 mg/L. The broth is mixed and held at room temperature or 4° C. for 6 hrs to allow complete utilization of the glucose in the media and the functioning of the flocculant. A coagulant may be added at this point. The fermentation broth is centrifuged (using a bench centrifuge—Avant J18 rotor Bechman Coulter, to remove cells and the EPS like compounds. This is followed by filtration of the supernatant using a 0.2 μm hollow fiber membrane filtration to remove host protein and cell debris. The protein solution s then concentrated using a 5 kDa ultrafiltration membrane to over 30 g/L protein and diafiltered extensively to remove most of the organic and inorganic impurities. The resulting mixture is either microfiltered or heat treated for the final microbial reduction step pre drying. Steps of this method is illustrated in FIG. 4.

### Example 6: Use of Enzymes to Digest EPS

A Pichia Pastoris strain is grown in fermentation conditions in high-density growth conditions at about pH 5. After about 36 hours of growth under fermentation conditions, the pH is raised to about pH 6, and expression of a protein of interest is induced by the addition of methanol to the culture. At this point an EPS degrading enzyme such as glucanase, is added to the reactor. This will degrade the EPS molecules as they are secreted in the system. The final fermentation broth is centrifuged (using a bench centrifuge—Avant J18 rotor Bechman Coulter, to remove cells. This is followed by filtration of the supernatant using a 0.2 μm hollow fiber membrane filtration to remove host protein and cell debris. The protein solution is then concentrated using a 5 kDa ultrafiltration membrane to over 30 g/L protein and diafiltered extensively to remove most of the organic and inorganic impurities. The EPS degrading enzyme is removed with the biomass in centrifuge and/or in the MF step. The resulting mixture is either microfiltered or heat treated for the final microbial reduction step pre drying. Steps of this method is illustrated in FIG. 2.

### Example 7: Use of Adsorbent to Extract EPS

Pichia Pastoris strain is grown in fermentation conditions in high-density growth conditions at about pH 5. After about 36 hours of growth under fermentation conditions, the pH is raised to about pH 6, and expression of the protein of interest is induced by the addition of methanol to the culture. At the end of the fermentation, the broth is mixed and held at room temperature or 4ºC for 6 hrs to allow complete utilization of the glucose in the media. The fermentation broth is centrifuged (using a bench centrifuge—Avant J18 rotor Bechman Coulter, to remove cells. The centrate from the centrifuge is then passed on a filter precoated with the adsorbent to remove the EPS like compounds. If a filter is used, then the adsorbent is suspended in water and passed over the filter creating a uniform layer on the filter. This is followed by the passage of the centrate on the filter. This can also be done with a column packed with the same adsorbent. This step is followed by filtration of the supernatant using a 0.2 μm hollow fiber membrane filtration to remove host protein and cell debris. The protein solution is then concentrated using a 5 kDa ultrafiltration membrane to over 30 g/L protein and diafiltered extensively to remove most of the organic and inorganic impurities. The resulting mixture is either microfiltered or heat treated for the final microbial reduction step pre drying. Steps of this method is illustrated in FIG. 3.

### Example 8: Removal of Unfavorable Characteristics

A protein concentrate, obtained in the method described in Example 2, was then precipitated by adding ammonium sulfate to reach about 40% w/v concentration in the concentrate solution. The precipitate was then centrifuged (using a bench centrifuge—Avant J18 rotor, Bechman Coulter) to remove the protein. The protein was then resuspended using DI water to a 10% w/v solution. This solution is then diafiltered with DI water to a final conductivity of less than 1 mS/cm. The diafiltered protein is then microfiltered through a 0.2 μm membrane and freeze dried. These proteins are referred to as the “small scale preparations”.

For large scale fermentation, at the end of fermentation, the broth was chilled to 8° C. to slow the metabolism of the yeast. Prior to centrifugation, the broth was diluted to reach a packed cell volume of 25% v/v. The yeast cells were then removed by centrifugation and supernatant was stored and moved to the next step. This step is completed within 8 hours of completing the fermentation and can begin before chilling is complete. An appropriate disc stack centrifuge with large enough surface area and solid capacity is used for this purpose. The bulk OD600 measurement of the supernatant to indicate good separation is preferably <0.9 AU. The centrate was collected for further clarification through the 0.2 μm filtration. The 0.2 μm filtration will run in a tangential flow mode with both concentration and dia-filtration steps. During the concentration step, the retentate volume is reduced by about 6-9×. To achieve higher yields the retentate is continuously diafiltered with ten diavolumes of water. The permeate from the 0.2 μm TFF is concentrated 6-8× from its initial volume to around 50 g/L protein concentration. The final retentate was a dark green in color. This retentate was then diafiltered with 6-8 DVs of DI water. The diafiltered retentate is then sterile filtered using a 0.2 μm MF filter and spray dried with inlet temp around 165° C. and outlet temp not exceeding 80° C. The membranes may be hydrophilic polyethylene sulfone designed for protein applications. The temperature was maintained at 10° C. throughout the process. These proteins are referred to as the “large scale preparations”.

The above processes result in a dark green solution predrying. To decolorize and deodorize it, an oxidation step may be utilized. The pH of the retentate before diafiltration was reduced to 4 using 85% v/v phosphoric acid. Then, 35% v/v hydrogen peroxide was added slowly to saturate the final solution to a 3% v/v hydrogen peroxide mixture. The mixture was then held in a tank for 6 hrs while mixing slowly to prevent foam outs. The pH was then changed back to 6 using concentrated sodium hydroxide followed by the diafiltration, sterile filtration and drying. These protein samples are referred to as the “Example A” samples.

The samples from the end of fermentation and end of the process of Example A were analyzed using GC-MS. The prominent flavor and odor compounds observed are listed in the Table 6, below:

TABLE 6
List of prominent flavor/odor causing compounds at end of fermentation and purification.

| | Chemical | | | solubility | |
|---|---|---|---|---|---|
| Source | class | Compound | BP | alcohols | Typical source/use |
| Observed at | Alcohol | 1-Hexanol, 2-ethyl- | no-180 C. | yes | common in plant fruit wines |
| end of | | Benzyl alcohol | no-205 C. | yes | common in plant fruit wines |
| fermentation | Aldehydes | 2-Propanone | yes 56 C. | yes | acetone-industrial solvent |
| | Ketone | 2-Butanone | no- | yes | Industrial solvent |
| | | | azeotrope | | |
| | | 1-Dodecene | yes-79 C. | yes | known Fermentation volatile |
| | Ester | Propanoic acid, | no-249 C. | yes | known Fermentation volatile |
| | | 2-methyl-, | | | |
| | | 3-hydroxy-2, | | | |
| | | 4,4-trimethylpentyl | | | |
| | | ester | | | |
| Observed | Aldehydes | Butanal, 3-methyl- | yes-94 C. | yes | Isovaleraldehyde |
| post | Ketone | 2-Undecanone | no-231 C. | yes | common in plant fruit wines |
| purification | Alcohol | Benzene ethanol | no-221 C. | yes | phenylethanol-auto antibiotic |
| | | | | | produced by fungi |
| step | Acid | Nonanoic acid | no-254 C. | yes | pelargonic acid-herbicide; |
| | | | | | rancid odor, treatment of seizures |
| | Aromatics | chlorotoluene | no-162 C. | yes | antibacterial irritating flavor |

The resulting protein retentate from the small-scale preparation and the large scale preparations was adjusted for pH and loaded on to a chromatography column. This column was packed with Cation exchange resin (SP400, Mitsubishi Chemicals, Japan). The chromatography steps were carried out with an AKTA Explorer 900 (GE Healthcare Life Sciences) and the Unicorn interface software (version 5.11) at approximately 22° C. in a down-flow mode, the chromatography method generally consisted of an equilibration step, a load (flowthrough) step, a wash step to remove non-bound protein, an elution step to remove product, a cleaning in place (CIP) step and regeneration step. The column volumes of elution, at each step and the buffer used is shown in the above Table 4 and the elution profile is shown in FIG. 12.

This elution fraction from the column is then concentrated and diafiltered to remove the elution buffer salts. This stream is then microfiltered using a 0.2 μm membrane and dried. These samples are referred to as the “Example B” samples.

The oxidized samples and non-oxidized samples were then tested with GC-MS using a polar, Stabilwax-DA column. The sample prep used was SPME and the analysis consisted of the MS data as well as the human olfactory odor testing. See Table 7, below for results for the small-scale protein preparations:

TABLE 7
GC MS data for samples generated in the lab setup (small scale
preparations) using oxidation and chromatography methods.

| compound | odor property | Example A | Example B |
|---|---|---|---|
| 2-methylpropanal | dark chocolate, malty | Weak | Very weak |
| 2-methylbutanal | dark chocolate, malty | Very weak | Weak |
| 3-methylbutanal | dark chocolate, malty | Weak | Medium |
| 2,3-butanedione | buttery, creamy | Weak | Very weak |
| 1-hexen-3-one | skunky, rubbery, plastic | Weak | Weak |
| 1-octen-3-one | earthy, mushroom | Medium | Weak |
| “unknown” | milky, cooked milk, potato | Weak | Very weak |
| (E)-2-nonenal | stale, green, hay | Very weak | Medium |
| p-cresol | animal stable, barnyard | Very weak | Very weak |
| acetaldehyde | pungent, ethanolic | Very weak | Very weak |

See Table 8, below for results for the large-scale protein preparations:

TABLE 8
GC MS data for samples generated in the lab setup (small scale
preparations) using oxidation and chromatography methods.

| compound | odor property | Example A | Example B |
|---|---|---|---|
| 2-methylpropanal | dark chocolate, malty | Strong | Medium |
| 2-methylbutanal | dark chocolate, malty | Strong | Medium |
| 3-methylbutanal | dark chocolate, malty | Medium | Weak |
| 2,3-butanedione | buttery, creamy | Medium | Weak |
| 1-hexen-3-one | skunky, rubbery, plastic | Weak | Weak |
| 1-octen-3-one | earthy, mushroom | Medium | Weak |
| “unknown” | milky, cooked milk, potato | Weak | Weak |
| (E)-2-nonenal | stale, green, hay | Very weak | Weak |
| p-cresol | animal stable, barnyard | Weak | Very weak |
| acetaldehyde | pungent, ethanolic | Weak | Very weak |

Most of the compounds listed in Table 7 are reduced when comparing the Example A samples to the Example B samples. This is even clearer in Table 8, with the large scale preparations.

The Example B samples generated above were further reprocessed using various treatments to check the improvement in sensory characteristics. The treatments tested were as follows:
Ethanol wash: The spray dried protein powder was resuspended in 10% v/v ethanol solution to reach a solids concentration of 50 g/L. The solution was stirred for 1 hour at ambient temperature using a magnetic stirrer and then diafiltered with 4-5 DVs of DI water on a 5 kDa membrane. The retentate was sterile filtered and dried.
Ion exchange (IEX): The spray dried protein powder was resuspended in DI water to reach a solids concentration of 50 g/L. The process described in example 5 was repeated with this protein solution.
Heat and Vacuum: The spray dried protein powder was resuspended in DI water to reach a solids concentration of 50 g/L. The protein solution was then heated to 50-58 C and maintained for 1 hr under low vacuum (75-150 torr). The solution was then sterile filtered and dried.
IEX, heat and vacuum: This was an orthogonal approach to combine various purification methods together. The product from the example 5 was then resuspended in DI water to reach a solids concentration of 50 g/L. The protein solution was then heated to 50-58 C and maintained for 1 hr under low vacuum (75-150 torr). The solution was then sterile filtered and dried.

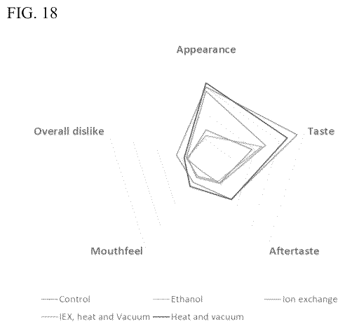

The sensory analysis performed on these samples based on appearance, mouthfeel and aftertaste of a 6% w/v solution in water was analyzed in FIG. 18. The ion exchange process of the non-oxidized preparations yielded the best version of the reprocessed control. The center of the graph represents the sensory profile of water.

### Example 9: Use of a Resin in the Bioreactor for Preparing a Protein Product Having a Reduced Quantity of a Recombinant Cell Byproduct

A method of the present disclosure is used to prepare a protein product having a reduced quantity of a recombinant cell byproduct. In this example, a recombinant egg-white protein is used.

Pichia Pastoris strain derived from the historic Phillips Petroleum strain NRRL Y-11430 is designed to express an illustrative protein, here ovomucoid. These transformant strains are further modified by adding a surface display enzyme that reduce the complex carbohydrate to filterable size. Sequencing confirms the strain does not contain any antibiotic markers or prokaryotic vector origin of replication sequences.

The resulting strain is grown in fermentation conditions in high-density growth conditions at about pH 5. After about 36 hours of growth under fermentation conditions, expression of the illustrative protein is induced by the addition of methanol to the culture.

An anionic exchange resin (e.g., DIAION HPA25L, Mitsubishi Chemicals, Japan) is pumped into the bioreactor. Prior to addition to the reactor, the anion exchange resin is sanitized with methanol, washed, and equilibrated with 25 mM citric acid buffer. The anionic resin in the reactor will absorb protein secreted by the Pichia culture and float to the top of the culture.

As the titer of the supernatant in the culture exceeds 50 g/L, there may be solubility-based issues with the stability of the system. The anionic resin will absorb protein to keep the system below the solubility limit.

The resin is withdrawn from the top of the fermenter and washed with DI water to remove components of the culturing medium. The pH is raised to 7 and the salt concentration adjusted to 300 mM NaCl to elute the protein. The protein is then diafiltered, followed by sterile filtration and drying.

The resin can then be autoclaved and reintroduced into the reactor to repeat the harvesting process. This process can be repeated multiple times, so long as the sterility of the system is maintained. The final harvest of the reactor occurs when the amount of dead cells or the cell biomass concentration interferes with the purification process due to the density and viscosity of the system. Alternately, the final harvest will occur when the expressed protein yield (e.g., protein titer) reduces to below an undesirable level.

### Example 10: Odoriferous Compounds in Egg Proteins Secreted from Yeast and Fungi

Proteins derived from the fermentation and downstream processing of yeast and fungi transformed to express an illustrative protein (e.g., ovomucoid (OVD) or ovalbumin (OVA)) may have contain off-flavor components. The following example illustrates the relative amounts of various typical aldehydes and acids eluted by the three isolates. This gives a relative comparison of two proteins—ovomucoid and ovalbumin—generated from the same base strain with respective genetic modifications. Also, this example demonstrates the superior odor profile of the ovalbumin protein generated using our platform yeast strain compared to a typical Aspergillus niger based strain.

The method used to generate the protein isolates from the three strains (A. niger based ovalbumin, P. pastoris based ovalbumin, P. pastoris based ovomucoid) is outlined. The wild strain was transformed to generate version to express the illustrative protein. For example, P. pastoris strain derived from strain NRRL Y-11430 was first designed to generate a nonmethanol-utilization (mutM) phenotype. These strains were then transformed to add in a strong methanol inducer promoter and to express an illustrative protein. The transformant strains were further modified by adding a surface display enzyme that would reduce the complex carbohydrate to filterable size, Sequencing confirmed that this strain did not contain any antibiotic markers or prokaryotic vector origin of replication sequences. The resulting strain was grown in fermentation conditions in high-density growth conditions at about pH 5. After about 36 hours of growth under fermentation conditions to generate high cell density, the pH was raised to about pH 6, and expression of the illustrative protein was induced by the addition of methanol to the culture. The fermentation broth was centrifuged (e.g., using a bench centrifuge—Avant J18 rotor Bechman Coulter) to remove cells. The centrifuged light phase or supernatant was filtered using a 0.2 μm hollow fiber membrane filtration to remove host protein and cell debris. The protein solution was then concentrated using a 5 kDa ultrafiltration membrane to over 30 g/L protein and diafiltered extensively to remove most of the organic and inorganic impurities. This concentrate was then microfiltered to remove the bioburden (e.g., contaminants) and dried using a freeze drier to generate the protein isolate powder.

Three protein isolates were generated from yeast (OVD or OVA) or fungi (OVD) and analyzed by GC-MS using a polar Stabilwax-DA column. The sample prep used was SPME and the analysis consisted of the MS data as well as human olfactory odor testing. Prominent off-flavor compounds observed are listed in Table 9.

TABLE 9
Concentration of various odorous aldehydes and acids generated by the
ovalbumin (OVA) and ovomucoid (OVD) protein isolates from yeast
and fungi source expressed in parts per billion (ppb) concentration

| | | | | |
|---|---|---|---|---|
| | | OVD | OVA | OVA |
| Compound | Odor Property | (yeast) | (yeast) | (fungi) |

Aldehydes

| | | | | |
|---|---|---|---|---|
| benzaldehyde | bitter almond | 1 | 1 | 7 |
| 2-methylbutanal | dark chocolate, malty | 5 | — | 10 |
| 3-methylbutanal | dark chocolate, malty | 2 | — | 11 |
| acetaldehyde | pungent, ethanolic | 6 | — | 9 |
| phenylacetaldehyde | musty | — | — | 8 |

Acids

| | | | | |
|---|---|---|---|---|
| acetic acid | vinegar | 4 | 2 | 1 |
| propanoic acid | rancid sweaty | — | — | 5 |
| 2-methylpropanoic | rancid butter | 9 | 5 | 6 |
| acid | | | | |
| butanoic acid | rancid butter | 7 | 3 | 2 |
| 2-methylbutanoic | rancid cheesy | 8 | 4 | — |
| acid | | | | |
| 3-methylbutanoic | rancid cheesy | 3 | — | — |
| acid | | | | |
| pentanoic acid | dirty socks | — | — | 4 |
| hexanoic acid | fatty, cheesy, waxy, | 10 | 6 | 3 |
| | barnyard animals | | | |

As shown in Table 9, there is a clear difference in the generation of aldehyde off-flavor components between the two proteins secreted by the yeast platform. With the exception of benzaldehyde, the yeast OVA system does not generate much aldehyde, whereas the yeast OVD system generates many aldehyde compounds.

### Example 11: Purifying Spray-Dried Protein Concentrate Using a Cationic Resin

Proteins derived from the fermentation and downstream processing of yeast and fungi transformed to express an illustrative protein (e.g., ovomucoid (OVD) or ovalbumin (OVA)) may contain off-flavor components and exopolysaccharide (EPS) due to the purification method. Powder proteins can be further purified to eliminate off-flavor and EPS compounds. The following example describes one such method.

P. pastoris strain derived from strain NRRL Y-11430 was first designed to generate a nonmethanol-utilization (mutM) phenotype. These strains were then transformed to add in a strong methanol inducer promoter and to express an illustrative protein. The transformant strains were further modified by adding a surface display enzyme that would reduce the complex carbohydrate to filterable size. Sequencing confirmed that this strain did not contain any antibiotic markers or prokaryotic vector origin of replication sequences. The resulting strain was grown in fermentation conditions in high-density growth conditions at about pH 5. After about 36 hours of growth under fermentation conditions to generate high cell density, the pH was raised to about pH 6, and expression of the illustrative protein was induced by the addition of methanol to the culture. The fermentation broth was centrifuged (e.g., using a bench centrifuge—Avant J18 rotor Bechman Coulter) to remove cells. The centrifuged light phase or supernatant was filtered using a 0.2 μm hollow fiber membrane filtration to remove host protein and cell debris. The protein solution was then concentrated using a 5 kDa ultrafiltration membrane to over 30 g/L protein and diafiltered extensively to remove most of the organic and inorganic impurities. This concentrate was then microfiltered to remove the bioburden (e.g., contaminant) and dried using a freeze drier to generate the protein isolate powder.

Isolate powder is resuspended in water in a ratio of 1 part protein to 10 parts DI water. The protein slurry is well mixed and diafiltered using a 5 kDa ultrafiltration membrane to achieve the desired conductivity for the operation of an ion exchange column. The protein concentrate is then adjusted to pH 3.5 using citrate and loaded on to a chromatography column packed with Cation exchange resin (SP400, Mitsubishi Chemicals, Japan). Chromatography steps are carried out using an AKTA Explorer 900 (GE Healthcare Life Sciences) and Unicorn interface software (version 5.11) at approximately 22° C. in a down-flow mode. An exemplary chromatography method comprises an equilibration step, a load (flowthrough) step, a wash step to remove non-bound protein, an elution step to remove product, a cleaning in place (CIP) step, and a regeneration step. The column volumes of elution, at each step and the buffer used is shown in Table 10.

TABLE 10
List of buffers and the column volumes
required in a typical process

| | Number of | |
|---|---|---|
| | Column | |
| Step | Volumes | Buffer composition |
| Equilibration | 4 | 25 mM Citrate buffer |
| Sample | 1-2 | Feed sample |
| application | | |
| Column Wash | 2-3 | 25 mM Citrate buffer |
| Elution | 3-4 | 25 mM Citrate buffer |
| | | with 1 M NaCl |
| CIP | 3 | 1 M NaOH |
| Regeneration | 3 | 20% EtOH and |
| | | 150 mM NaCl |

The elution fraction was then concentrated to over 30 g/L concentration and diafiltered using a 5 kDa membrane. This concentrate was then micro filtered to remove the bioburden and dried using a freeze drier to generate the purified protein isolate powder.

### Example 12: Purifying Spray Dried Protein Concentrate Using an Anionic Resin

Isolate powder generated with the procedure outlined in Example 11 is resuspended in water in a ratio of 1 part protein to 10 parts DI water. The protein slurry is well mixed and diafiltered using a 5 kDa ultrafiltration membrane to achieve the desired conductivity for the operation of an ion exchange column. The protein concentrate is then adjusted to pH 6 using phosphate buffer and loaded on to a chromatography column packed with Anion exchange resin capto Q resin (Cytiva Chemicals). The buffer compositions and the column volumes are maintained as shown in Table 11. Column packing and chromatography steps are carried out using an AKTA Explorer 900 (GE Healthcare Life Sciences) and Unicorn interface software (version 5.11) at approximately 22° C. in a down-flow mode. An exemplary chromatography method comprises an equilibration step, a load (flowthrough) step, a wash step to remove non-bound protein, an elution step to remove product, a cleaning in place (CIP) step, and a regeneration step.

The elution fraction was then collected, concentrated to over 30 g/L concentration and diafiltered using a 5 kDa membrane. This concentrate was then micro filtered to remove the bioburden and dried using a freeze drier to generate the protein isolate powder.

Sensory analysis was performed based on appearance, mouthfeel, and aftertaste of a 6% w/v solution in water. The results were summarized in FIG. 18.

TABLE 11
List of buffers and the column volumes
required in a typical process.

| | Number of | |
|---|---|---|
| | Column | |
| Step | Volumes | Buffer composition |
| Equilibration | 4 | 25 mM Sodium Phosphate + |
| | | 16 mM Sodium Chloride (pH 6; |
| | | Conductivity-3.4 mS/cm) |
| Sample | 1-2 | Feed sample diluted to 3.4 mS/cm |
| application | | |
| Column Wash | 2-3 | Same as equilibration |
| Elution | 3-4 | 25 mM Sodium Phosphate + |
| | | 300 mM Sodium Chloride |
| CIP | 3 | 1 M NaOH |
| Regeneration | 3 | 20% EtOH and 16 mM NaCl |

### Example 13: Purifying Spray Dried Protein Concentrate Using Oxidation

The process in Example 11 will produce an odoriferous solution depending on the source platform and the prior purification method used. To decolorize and deodorize the solution, an oxidation step may be utilized.

Isolate powder generated with the procedure outlined in Example 11 was resuspended in water in a ratio of 1 part protein to 10 parts DI water. The protein slurry was well mixed and the pH was reduced to pH4 using 85% v/v phosphoric acid. 35% v/v hydrogen peroxide was then added slowly to saturate the final solution to a 3% v/v hydrogen peroxide mixture. The mixture was then held in a tank for 6 hours while mixing slowly to prevent foam outs. The solution was then changed back to pH 6 using concentrated sodium hydroxide followed by diafiltration using a 5 kDa ultrafiltration membrane, micro filtration and drying to obtain purified protein isolate.

Sensory analysis was performed based on appearance, mouthfeel, and aftertaste of a 6% w/v solution in water. The results were summarized in FIG. 18.

### Example 14: Purifying Spray Dried Protein Concentrate Using an Amphipathic Solvent

The process in Example 11 will produce an odoriferous solution depending on the source platform and the prior purification method used. To decolorize and deodorize the solution, an oxidation step may be utilized.

Isolate powder generated with the procedure outlined in Example 11 was resuspended in 10% v/v ethanol solution to reach a solid concentration of 50 g/L. The solution was stirred for 1 hour at ambient temperature using a magnetic stirrer and then diafiltered with 4-5 DVs of DI water on a 5 kDa membrane. The retentate was sterile filtered and dried to obtain purified protein isolate.

Sensory analysis was performed based on appearance, mouthfeel, and aftertaste of a 6% w/v solution in water. The results were summarized in FIG. 18.

### Example 15: Purifying Spray Dried Protein Concentrate Using Heat and Vacuum

The process in Example 11 will produce an odoriferous solution depending on the source platform and the prior purification method used. To decolorize and deodorize the solution, an oxidation step may be utilized.

Isolate powder generated with the procedure outlined in Example 11 was resuspended in water in a ratio of 1 part protein to 10 parts DI water. The protein slurry was well mixed The protein slurry is then heated to 80° C. and maintained for 1 hour under low vacuum (e.g., 75-150 torr). The solution was then sterile filtered and dried to obtain purified protein isolate. In case of an ovalbumin protein isolate, the solution cannot be heated to a temperature more than 65° C.

Sensory analysis was performed based on appearance, mouthfeel, and aftertaste of a 6% w/v solution in water. The results were summarized in FIG. 18.

### Example 16: Purifying Spray Dried Protein Concentrate Using Diafiltration and Ultrafiltration

The process in Example 11 will produce an odoriferous solution depending on the source platform and the prior purification method used. To decolorize and deodorize the solution, an oxidation step may be utilized.

Isolate powder generated with the procedure outlined in Example 11 was resuspended in water in a ratio of 1 part protein to 20 parts DI water. The protein slurry was well mixed and then diafiltered with 4-5 DVs of DI water on a 5 kDa membrane. The retentate was filtered through a series of filters. The first in series being the carbon impregnated filter on a coarse 1 μm microfilter followed by a coarse 1 μm filter and finally a 0.2 μm filter. This solution was then dried with a spray drier to obtain purified protein isolate.

Sensory analysis was performed based on appearance, mouthfeel, and aftertaste of a 6% w/v solution in water. The results were summarized in FIG. 18.

### Example 17: Purifying Spray-Dried Protein Concentrate Using a Low Fouling Filtration Membrane and Dry Isolate

Proteins derived from the fermentation and downstream processing of yeast and fungi transformed to express an illustrative protein (e.g., ovomucoid (OVD) or ovalbumin (OVA)) may contain off-flavor components and exopolysaccharide (EPS) due to the purification method. Powder proteins can be further purified to eliminate off-flavor and EPS compounds. The following example describes one such method. The concentration membranes typically used create a substantial challenge in terms of operation and product consistency due to fouling. To maintain a consistent product, low cost of cleaning, simpler operation, we tried a low fouling membrane from (Zwitterco Inc.) was used.

The low fouling membrane is synthesized with a zwitterionic surface coat that enables the water permeation, repels the protein from the surface and minimizes the concentration polarization layer formation. This takes the advantage of the zwitterionic surfaces enabling the flux to be constant throughout the process. Water-loving zwitterions are combined with a strong, hydrophobic backbone to get highly hydrophilic, net-neutral surface and pore structure that is highly resistant to fouling from organic molecules (e.g. fats, oils, proteins). This chemistry results in intrinsic pore uniformity, no ionic or other interactions at the surface, and high tolerance of salts, pH.

As a result, these membranes deliver consistent and higher flux than the conventional membrane and retain flux with mild maintenance washes without any irreversible fouling. Further, high consistent flux increases the concentration factor and enables case of implementing the continuous system. Also, a reduced or a milder cleaning directly results in the increase of the membrane life thereby reducing the operating costs substantially (chemicals, membranes) as well a greener process with lower wastewater burden on the system. Methods using low fouling filtration for the preparation of purified recombinant protein concentrate or dried recombinant protein are illustrated in FIGS. 19 and 20.

Isolate powder generated with the procedure outlined in Example 11 was resuspended in water in a ratio of 1 part protein to 20 parts DI water to about 5% w/v solution. The protein slurry was well mixed, concentrated using a low fouling ultrafiltration membrane (Zwitterco Inc) and then diafiltered with 4-5 DVs of DI water on a 5 kDa membrane. The retentate was filtered through a series of filters. The first in series being the carbon impregnated filter on a coarse 1 μm microfilter followed by a coarse 1 μm filter and finally a 0.2 μm filter. This solution was then dried with a spray drier to obtain purified protein isolate.

Sensory analysis was performed based on appearance, mouthfeel, and aftertaste of a 6% w/v solution in water.

### Example 18: Purifying Spray-Dried Protein Concentrate Using a Low Fouling Filtration Membrane

Proteins derived from the fermentation and downstream processing of yeast and fungi transformed to express an illustrative protein (e.g., ovomucoid (OVD) or ovalbumin (OVA)) may contain off-flavor components and exopolysaccharide (EPS) due to the purification method. Powder proteins can be further purified to eliminate off-flavor and EPS compounds. The following example describes one such method. The concentration membranes typically used create a substantial challenge in terms of operation and product consistency due to fouling. To maintain a consistent product, low cost of cleaning, simpler operation, a low fouling membrane (Zwitterco Inc) was used.

P. pastoris strain derived from strain NRRL Y-11430 was first designed to generate a non methanol-utilization (mutM) phenotype. These strains were then transformed to add in a strong methanol inducer promoter and to express an illustrative protein, ovalbumin. The transformant strains were further modified by adding a surface display enzyme that would reduce the complex carbohydrate to filterable size. Sequencing confirmed that this strain did not contain any antibiotic markers or prokaryotic vector origin of replication sequences. The resulting strain was grown in fermentation conditions in high-density growth conditions at about pH 5. After about 36 hours of growth under fermentation conditions to generate high cell density, the pH was raised to about pH 6, and expression of the illustrative protein was induced by the addition of methanol to the culture. The fermentation broth was diluted to reach a packed cell volume of 25% v/v. The yeast cells were then removed by centrifugation and supernatant was stored and moved to the next step. The bulk OD600 measurement of the supernatant to indicate good separation is preferably <0.9 AU. The concentrate was collected for further clarification through the 0.2 μm filtration. The 0.2 μm filtration will runs in a tangential flow mode with both concentration and dia-filtration steps. The protein solution was then split into two equal volumes. One part was concentrated using a typical large scale 10 kDa ultrafiltration membrane (manufactured by Synder Filtration or Koch Industries or Alfalaval Inc or Cytiva Inc) to over 30 g/L protein and diafiltered extensively to remove most of the organic and inorganic impurities. The other part was concentrated and diafiltered using a “0 fouling membrane” (Zwitterionic membrane from Zwitterco Inc). This concentrate was then individually microfiltered to remove the bioburden (e.g., contaminants). This was achieved by passing the concentrate through a series of filters. The first in series being the carbon impregnated filter on a coarse 1 μm microfilter followed by a coarse 1 μm filter and finally a 0.2 μm filter. The product held at 4 C for 48 hours until it was spray dried to obtain protein isolate powder.

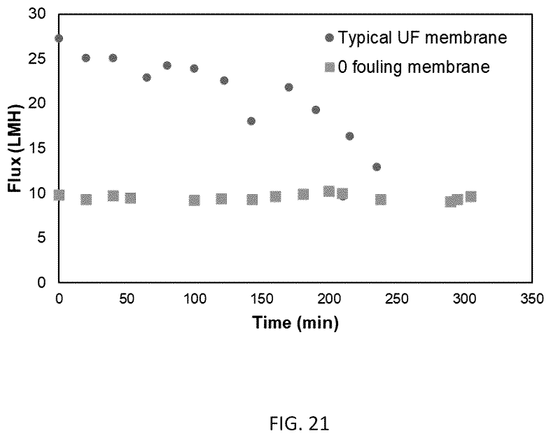

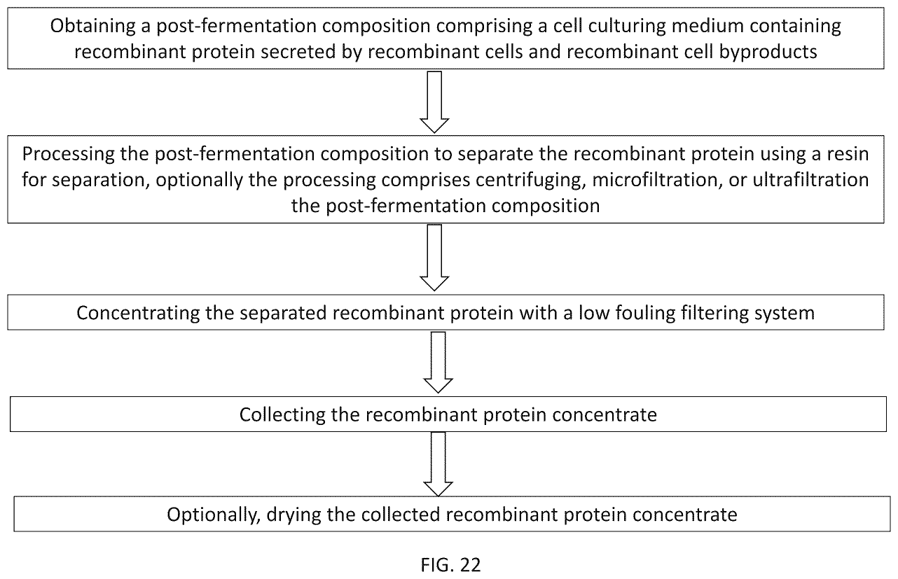

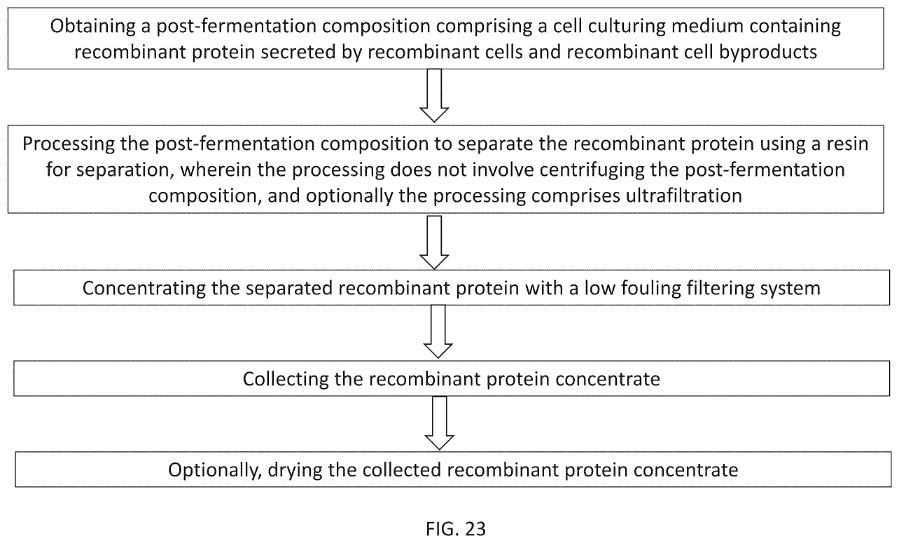

FIG. 21 compares the performance of the 0 fouling membrane to a typical membrane. FIG. 21 shows flux curves comparing the performance of the typical ultrafiltration (UF) membrane and the zwitterionic membrane (0 fouling membrane) over time. The zwitterionic membrane (0 fouling membrane) demonstrated stable flux throughout the concentration and diafiltration steps and was equal to the water flux. The membranes were compared at similar transmembrane pressure (TMP), which represents the average pressure across the membrane. However, the manufacturer recommends higher TMP which will potentially exceed the performance of the typical membrane but also will increase the capital expense as well. The data demonstrate that low fouling filtration can improve the quality and purification of fermentation compositions comprising secreted recombinant protein and the recombinant cells that secret the recombinant protein to obtain recombinant protein concentrate. FIGS. 22 and 23 illustrate exemplary methods for preparing the recombinant protein concentrate.

### Example 19: Purifying Spray-Dried Protein Concentrate Using a Filtration System Designed to Reach High Concentrations

Proteins derived from the fermentation and downstream processing of yeast and fungi transformed to express an illustrative protein (e.g., ovomucoid (OVD) or ovalbumin (OVA)) may contain off-flavor components and exopolysaccharide (EPS) due to the purification method. Powder proteins can be further purified to eliminate off-flavor and EPS compounds. The following example describes one such method. The concentration membranes typically used create a substantial challenge in terms of operation and product consistency due to fouling. To maintain a consistent product, low cost of cleaning, simpler operation, a low fouling system (SmartFlow Technologies Inc) was tested.

Isolate powder generated with the procedure outlined in Example 11 was resuspended in water in a ratio of 1 part protein to 20 parts DI water to about 5% w/v solution. The protein slurry was well mixed and passed through a series of filters. The first in series being the carbon impregnated filter on a coarse 1 μm microfilter followed by a coarse 1 μm filter and finally a 0.2 μm filter. The filtration unit was cleaned and steam sterilized. The slurry was then concentrated to a 40% w/v or 400 g/L solution using a low fouling ultrafiltration system. This solution was then dried with a spray drier to obtain purified protein isolate.

### Example 20: Liquid Protein Concentrate Product

Proteins derived from the fermentation and downstream processing of yeast and fungi transformed to express an illustrative protein (e.g., ovomucoid (OVD) or ovalbumin (OVA)) may contain off-flavor components and exopolysaccharide (EPS) due to the purification method. Powder proteins can be further purified to eliminate off-flavor and EPS compounds. The following example describes one such method. The concentration membranes typically used create a substantial challenge in terms of operation and product consistency due to fouling. To maintain a consistent product, low cost of cleaning, simpler operation, a low fouling system (SmartFlow Technologies Inc) was tested.

A liquid concentrate product may be highly desirable in applications that are designed in facilities with no powder handling systems. It makes aliquoting easier. The mixing is also simplified with minimal losses, safety hazards related to dust handling. However, the limitations with such systems can be shelf life and supply chain for liquid product distribution.

P. pastoris strain derived from strain NRRL Y-11430 was first designed to generate a non methanol-utilization (mutM) phenotype. These strains were then transformed to add in a strong methanol inducer promoter and to express an illustrative protein. The transformant strains were further modified by adding a surface display enzyme that would reduce the complex carbohydrate to filterable size. Sequencing confirmed that this strain did not contain any antibiotic markers or prokaryotic vector origin of replication sequences. The resulting strain was grown in fermentation conditions in high-density growth conditions at about pH 5. After about 36 hours of growth under fermentation conditions to generate high cell density, the pH was raised to about pH 6, and expression of the illustrative protein was induced by the addition of methanol to the culture. The fermentation broth was centrifuged (e.g., using a bench centrifuge—Avant J18 rotor Bechman Coulter) to remove cells. The centrifuged light phase or supernatant was filtered using a 0.2 μm hollow fiber membrane filtration to remove host protein and cell debris. The protein solution was then concentrated using a 5 kDa ultrafiltration membrane to over 30 g/L protein and diafiltered extensively to remove most of the organic and inorganic impurities. This concentrate was then microfiltered to remove the bioburden such as contaminants. This was achieved by passing the concentrate through a series of filters. The first in series being the carbon impregnated filter on a coarse 1 μm microfilter followed by a coarse 1 μm filter and finally a 0.2 μm filter. The filtration unit was cleaned and steam sterilized. The slurry was then concentrated to a 40% w/v or 400 g/L solution using a low fouling ultrafiltration system. This solution was then formulated to obtain a protein concentrate product. This product was aseptically packaged and distributed to the end users.

The protein concentrate can be formulated to make other liquid products such as liquid egg, which does not contain additives such as sugar, cream, milk, and/or sweetener. The protein concentrate can also be formulated to make beverages or gummy supplements.

Standard test SOPs were used for the protein concentrate and the product preparation. Surprisingly the sensory evaluation of the product using liquid protein concentrate was better than the control using egg white protein as shown Table 12.

TABLE 12
Sensory evaluation of the egg scramble prepared using Control
(egg white protein) and the liquid protein concentrate (LPC)

| | Appear- | | | | | Overall |
|---|---|---|---|---|---|---|
| | ance | Aroma | Taste | Texture | Mouthfeel | Liking |
| Control (EWP) | 5.6 | 4.8 | 4.5 | 5.2 | 5.0 | 4.9 |
| LPC | 5.9 | 4.8 | 5.2 | 5.5 | 5.3 | 5.3 |
| Difference | 0.3 | 0.0 | 0.7 | 0.3 | 0.3 | 0.4 |

Further, the texture analysis showed that the liquid egg performed better and we could possibly reduce the dosage to achieve the desired functionality as shown in Table 13.

TABLE 13
Functionality evaluation of the egg scramble prepared
using Control (egg white protein) and the
liquid protein concentrate (LPC)

| | | | Instant | |
|---|---|---|---|---|
| | Firmness | Toughness | Springiness | Resilience |
| Control | 12937 | 5972 | 6.73 | 37.34 |
| (EWP) | | | | |
| LPC | 15570 | 7609 | 6.64 | 34 |
| % Difference | 20.35% | 27.41% | −1.34% | −8.94% |
| from EWP | | | | |

This test was performed to understand the effect of holding conditions on the product performance. The protein concentrate was tested in the liquid form transported under refrigerated conditions as well as in frozen form transported under frozen conditions. The texture comparison was shown in the following table. Surprisingly, the frozen product performed substantially poorer than the liquid one indicating the preferred mode of transport and usage, as shown in Tables 14 and 15.

TABLE 14
Functionality evaluation of the product prepared
using Control (egg white protein) and the
liquid protein concentrate (LPC) in liquid form

| | | | Instant | |
|---|---|---|---|---|
| | Firmness | Toughness | Springiness | Resilience |
| Control | 8508 | 4721 | 24.9 | 0.303 |
| (EWP) | | | | |
| LPC (stored | 11559 | 5958 | 30.16 | 0.337 |
| at 4° C.) | | | | |
| % Difference | 35.86% | 26.20% | 21.12% | 11.22% |
| from EWP | | | | |

TABLE 15
Functionality evaluation of the product prepared
using Control (egg white protein) and the
liquid protein concentrate (LPC) in frozen form

| | Firmness | | Instant | |
|---|---|---|---|---|
| | (g) | Toughness | Springiness | Resilience |
| Control | 8508 | 4721 | 24.9 | 0.303 |
| (EWP) | | | | |
| LPC (stored | 17874 | 10778 | 37.2 | 0.344 |
| at −20° C.) | | | | |
| % Difference | 110.08% | 128.30% | 49.40% | 13.53% |
| from EWP | | | | |

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes. In particular, PCT/US2022/038074, U.S. application Ser. No. 18/050,213, U.S. Pat. No. 11,718,644; and U.S. application Ser. No. 18/336,915, each of which is herein incorporated by reference in its entirety.

What is claimed is:

1. A method for preparing a recombinant protein concentrate for a consumable food product, the protein product having a reduced quantity of recombinant cell byproducts, the method comprising:
obtaining a post-fermentation composition from a fermentation process used to produce recombinant egg protein, the post-fermentation composition comprising a cell culturing medium containing recombinant protein secreted by recombinant cells and recombinant cell byproducts;
processing the post-fermentation composition under conditions that separate the recombinant protein from the plurality of recombinant cell byproducts without using a resin for separation, thereby obtaining a separated recombinant protein;
concentrating the separated recombinant protein with a low fouling filtering system that filters the separated recombinant protein to produce a recombinant protein concentrate; and
collecting the recombinant protein concentrate comprising recombinant egg protein for an ingredient composition in a consumable food product.

2. The method of claim 1, wherein the recombinant protein concentrate has a concentration of at least 400 g/L.

3. The method of claim 1, wherein the consumable food product containing the recombinant protein concentrate has an at least 25% reduction in the quantity of exopolysaccharide(EPS) and/or a quantity of off-flavor components relative to the post-fermentation composition comprising the recombinant protein and the recombinant cell byproducts.

4. The method of claim 1, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

5. The method of claim 1, wherein the egg protein comprises ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

6. The method of claim 5, wherein the egg protein has a sequence that is at least 80% identical to the egg protein naturally produced in a bird, a chicken, quail, turkey, turkey vulture, hummingbird, duck, ostrich, goose, gull, guineafowl, pheasant, or emu.

7. The method of claim 1, wherein the consumable food product comprising the recombinant protein concentrate comprises liquid egg, beverages, baked food, dietary supplements, or gummy supplements.

8. The method of claim 1, wherein the recombinant protein concentrate provides an improvement to at least one additional feature selected from the group consisting of: flavor, moisture retention, water activity, mouthfeel, texture, hardness, cohesiveness, springiness, chewiness, stability to heat treatment, stability to pH, firmness, toughness, resilience, and stability for transportation.

9. A method for preparing an ingredient composition for a consumable food product comprising a recombinant protein concentrate, the method comprising:
processing a cell culturing medium comprising:
(i) recombinant cells that secrete a recombinant egg protein;
(ii) the recombinant egg protein secreted; and
(iii) recombinant cell byproducts,
under conditions that separate the recombinant egg protein from the recombinant cell byproducts without using a resin, thereby obtaining a separated recombinant protein having a reduced quantity of the recombinant cell byproducts for preparation of the consumable food product;
concentrating the separated recombinant protein with a low fouling filtering system that filters the separated recombinant protein to produce a recombinant protein concentrate;
collecting the recombinant protein concentrate comprising recombinant egg protein; and
preparing the ingredient composition comprising the recombinant protein concentrate for the consumable food product, the consumable food product comprising liquid egg, beverages, baked food, egg scrambles, dietary supplements, or gummy supplements.

10. The method of claim 9, wherein the consumable food product containing the recombinant protein concentrate has an at least 25% reduction in the quantity of exopolysaccharide(EPS) and/or a quantity of off-flavor components relative to the post-fermentation composition comprising the recombinant protein and the recombinant cell byproducts.

11. The method of claim 9, wherein the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum glocosporiodes, Endothia spp., Endothia parasitica, Escherichia coli, Fusarium spp., Fusarium gramincarum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor michei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

12. The method of claim 9, wherein the egg protein comprises ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.

13. The method of claim 9, wherein the recombinant protein concentrate provides an improvement to at least one additional feature selected from the group consisting of: flavor, moisture retention, water activity, mouthfeel, texture, hardness, cohesiveness, springiness, chewiness, stability to heat treatment, stability to pH, firmness, toughness, resilience, and stability for transportation.

14. A method for preparing a consumable composition, the method comprising:
obtaining a composition comprising a recombinant protein and a plurality of recombinant cell byproducts in a cell culture medium,
wherein the recombinant cell byproducts comprise an exopolysaccharide (EPS) and an off-flavor component;
contacting, in a bioreactor, the composition with an anionic resin that reversibly attaches to the recombinant protein and does not substantially attach to the plurality of recombinant cell byproducts;
separating the recombinant protein attached to the anionic resin from the plurality of recombinant cell byproducts;
collecting the separated recombinant protein, thereby obtaining a protein product having a reduced quantity of the plurality of recombinant cell byproducts; and
formulating a consumable composition comprising the protein product.

15. The method of claim 14, wherein the composition comprising the recombinant protein and the plurality of recombinant cell byproducts has a pH greater than the isoelectric point (pI) of the recombinant protein, or the pH of the composition comprising the recombinant protein and the plurality of recombinant cell byproducts is not modified to achieve a pH greater than the pI of the recombinant protein.

16. The method of claim 14, wherein the anion resin is selected from the group consisting of: Capto Q resin, a DEAE type weak anion exchanger, a resin with trimethyl aminoethyl groups, a resin with triethyl aminoethyl groups, a resin with quaternary amine groups, or combinations thereof.

17. The method of claim 14, wherein the anionic resin is reintroduced into the bioreactor after withdrawing the anionic resin from the bioreactor to separate the recombinant protein attached to the anionic resin.

18. The method of claim 14, further comprising a concentration step and/or diafiltration treatment of the separated recombinant protein to produce a recombinant protein-containing composition having a preferred pH and/or ionic condition.

19. The method of claim 14, further comprising heat treating and/or drying of the recombinant protein-containing composition to produce a dry protein product having a reduced quantity of the plurality of recombinant cell byproducts.

20. The method of claim 14, wherein the heat treatment separates the recombinant protein and the off-flavor component, wherein the heat is applied at a temperature and duration such that the off-flavor component is volatized and a gaseous off-flavor component is removable.

21. The method of claim 20, wherein a vacuum is applied contemporaneous with the application of heat and the vacuum facilitates removal of the gaseous off-flavor component.

22. The method of claim 14, wherein the off-flavor component is an acid, an alcohol, an aldehyde, an aromatic, an ester, or a ketone.

23. The method of claim 14, wherein the temperature of the protein-containing composition having a preferred pH and/or ionic conditions, the composition comprising the recombinant protein and the plurality of recombinant cell byproducts, and/or the protein product having a reduced quantity of the plurality of recombinant cell byproducts during the heat treatment is up to 80° C., or from about 50° C. to about 60° C.

24. The method of claim 14, wherein the composition comprising the recombinant protein and the plurality of recombinant cell byproducts and/or the protein product having a reduced quantity of the plurality of recombinant cell byproducts further undergoes an oxidation step involving the addition of hydrogen peroxide.

25. The method of claim 14, wherein the protein product has at least 25% reduction in the quantity of EPS and/or the quantity of off-flavor components relative to the composition comprising a recombinant protein and the plurality of recombinant cell byproducts.

26. The method of claim 14, wherein the recombinant cell that expresses the recombinant protein and the plurality of recombinant cell byproducts is selected from a fungal cell, such as filamentous fungus or a yeast, a bacterial cell, a plant cell, an insect cell, or a mammalian cell.

27. The method of claim 14, wherein the recombinant protein is an enzyme, a nutritive protein, a food ingredient, or a food additive.

28. The method of claim 27, wherein the enzyme is pepsinogen or pepsin.

29. The method of claim 27, wherein the protein is an egg-white protein.

30. The method of claim 29, wherein the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof.
